(19) United States
(12) Patent Application Publication
(10) Pub. No.: US 2024/0206796 A1
(43) Pub. Date: Jun. 27, 2024

(54) HEART'S ELECTROMOTIVE FORCE ESTIMATION METHOD, HEART'S ELECTROMOTIVE FORCE ESTIMATION APPARATUS AND PROGRAM

(71) Applicant: NIPPON TELEGRAPH AND TELEPHONE CORPORATION, Tokyo (JP)

(72) Inventor: Shingo TSUKADA, Musashino-shi, Tokyo (JP)

(73) Assignee: NIPPON TELEGRAPH AND TELEPHONE CORPORATION, Tokyo (JP)

(21) Appl. No.: 17/909,716

(22) PCT Filed: Mar. 9, 2020
(86) PCT No.: PCT/JP2020/010025

§ 371 Date: Sep. 6, 2022

(51) Int. Cl. A61B 5/346; A61B 5/329

(52) U.S. Cl. CPC A61B 5/346; A61B 5/329

(57) ABSTRACT

An electromotive force estimation method includes steps of: dividing a time series of an electrocardiographic potential to be estimated in a time series of an electromotive force into unit electrocardiographic potential time series such that time series representing electrocardiographic potentials generated by one pulsation of a myocardium are separated from each other; acquiring, for each unit electrocardiographic potential time series, a depolarization electrocardiographic potential time series that is a set of data belonging to a depolarization period, a refractory electrocardiographic potential time series that is a set of data belonging to a refractory period, and a repolarization electrocardiographic potential time series that is a set of data belonging to a repolarization period; estimating a depolarization electromotive force time series indicating an electromotive force at each time point in the depolarization period based on the depolarization electrocardiographic potential time series; estimating a refractory electromotive force time series indicating an electromotive force at each time point of the refractory period based on the refractory electrocardiographic potential time series and an estimation value of the electromotive force at an end time point of the depolarization period; and estimating a repolarization electromotive force time series indicating an electromotive force at each time point of the repolarization period based on the repolarization electrocardiographic potential time series and an estimation value of the electromotive force at an end time point of the refractory period.

### TECHNICAL FIELD

The present disclosure relates to an electromotive force estimation method, an electromotive force estimation device, and a program.

### BACKGROUND ART

It is known that abnormality of the myocardium due to myocardial ischemia typified by myocardial infarction and angina pectoris appears as abnormality of electromotive force that is a set of membrane potentials of the myocardium. Disorders of the myocardium and the stimulation conduction system and a load on the heart such as heart failure also affect the electromotive force. However, it is difficult to directly measure the electromotive force. Accordingly, in many cases in medical practice, abnormality of the myocardium is estimated by observing an electrocardiogram that indicates a time series of an electrocardiographic potential on the body surface generated by electromotive force. Specifically, the abnormality of the myocardium is estimated by observing each component of P, QRS, ST, and T in the time series of the electrocardiographic potential and a change in the interval. However, as compared with the electromotive force of an original signal, the electrocardiographic potential of the body surface significantly attenuates around the DC component, so that the change caused by the abnormality of the myocardium is small, and thus it may be difficult to detect the abnormality. Thus, a method of estimating a cardiac function including an electromotive force by executing a simulation using a biophysical or mathematical model that reproduces a time series of the electrocardiographic potential has been proposed.

### CITATION LIST

### Non Patent Literature

NPL 1: Toru Oe, “Cardiac Electrophysiological Test (Monophasic Action Potential Recording)”, Journal of JCS Cardiologists, Circulation Journal, Vol. 28, p. 129-135, 2019, August
NPL 2: K. H. W. J. ten Tusscher, D. Noble, P. J. Noble, and A. V. Panfilov, “A model for human ventricular tissue”, Am J Physiol Heart Circ Physiol. 286: H1573-H1589, 2004
NPL 3: Hiroshi Tanaka, “On the Inverse Solution of Electrocardiology”, Japanese Journal of Medical Electronics and Biological Engineering, 1985, Vol. 23, No. 3, p. 147-158
NPL 4: MICHAEL R. FRANZ, “Long-Term Recording of Monophasic Action Potentials From Human Endocardium” Am J Cardiol. 1983 June; 51, p. 1629-1634

### SUMMARY OF THE INVENTION

### Technical Problem

However, simulation such as a nonlinear finite element method using a biophysical or mathematical model has a high calculation load and requires experience and advanced technology to match with a measurement value. Consequently, it is necessary to prepare a computer with high performance and it takes time to obtain a result.

In addition, severe heart diseases such as myocardial infarction and fatal arrhythmia lead to death in a short time after abnormality occurs, and rapid abnormality detection and evaluation are required. However, since it is difficult to rapidly calculate the electromotive force in the related art, the abnormality cannot be determined until the electrocardiographic potential of the body surface exhibits a clear abnormality, and the disease state becomes severe.

In view of the above circumstances, an object of the present disclosure is to provide a technique for reducing a calculation load in estimating an electromotive force.

### Means for Solving the Problem

An aspect of the present disclosure is an electromotive force estimation method, including:
dividing an electrocardiographic potential time series to be estimated in a time series of an electromotive force such that time series representing electrocardiographic potentials generated by one pulsation of a myocardium are separated from each other;
acquiring, for each unit electrocardiographic potential time series, a depolarization electrocardiographic potential time series that is a set of data belonging to the depolarization period, a refractory electrocardiographic potential time series that is a set of data belonging to the refractory period, and a repolarization electrocardiographic potential time series that is a set of data belonging to the repolarization period by classifying pieces of data of the unit electrocardiographic potential time series into data belonging to the depolarization period, data belonging to the refractory period, data belonging to the repolarization period, and data belonging to a resting period for each unit electrocardiographic potential time series while setting each time series divided in the dividing as a unit electrocardiographic potential time series;
estimating a depolarization electromotive force time series indicating an electromotive force at time points in the depolarization period based on the depolarization electrocardiographic potential time series;
estimating a refractory electromotive force time series indicating an electromotive force at time points of the refractory period based on the refractory electrocardiographic potential time series and an estimation value of the electromotive force at an end time point of the depolarization period; and
estimating a repolarization electromotive force time series indicating an electromotive force at time points of the repolarization period based on the repolarization electrocardiographic potential time series and an estimation value of the electromotive force at an end time point of the refractory period.

### Effects of the Invention

The present disclosure enables a reduction of the calculation load in estimating the electromotive force.

### BRIEF DESCRIPTION OF DRAWINGS

FIG. 1 is an explanatory diagram for explaining an outline of an electromotive force estimation method of an embodiment.

FIG. 2 is a flowchart illustrating an example of a flow of processing executed in the electromotive force estimation method of the embodiment.

FIG. 3 is a flowchart illustrating an example of a processing flow of estimating main unit electromotive force time series according to the embodiment.

FIG. 4 is a first explanatory diagram in which the measured electrocardiographic potential time series is used for illustrating the certainty of the estimation result by the electromotive force estimation method of the embodiment.

FIG. 5 is a second explanatory diagram in which the measured electrocardiographic potential time series is used for illustrating the certainty of the estimation result by the electromotive force estimation method of the embodiment.

FIG. 6 is a third explanatory diagram in which the measured electrocardiographic potential time series is used for illustrating the certainty of the estimation result by the electromotive force estimation method of the embodiment.

FIG. 7 is a fourth explanatory diagram in which the measured electrocardiographic potential time series is used for illustrating the certainty of the estimation result by the electromotive force estimation method of the embodiment.

FIG. 8 is a fifth explanatory diagram in which the measured electrocardiographic potential time series is used for illustrating the certainty of the estimation result by the electromotive force estimation method of the embodiment.

FIG. 9 is a sixth explanatory diagram in which the measured electrocardiographic potential time series is used for illustrating the certainty of the estimation result by the electromotive force estimation method of the embodiment.

FIG. 10 is a seventh explanatory diagram in which the measured electrocardiographic potential time series is used for illustrating the certainty of the estimation result by the electromotive force estimation method of the embodiment.

FIG. 11 is an eighth explanatory diagram in which the measured electrocardiographic potential time series is used for illustrating the certainty of the estimation result by the electromotive force estimation method of the embodiment.

FIG. 12 is a ninth explanatory diagram in which the measured electrocardiographic potential time series is used for illustrating the certainty of the estimation result by the electromotive force estimation method of the embodiment.

FIG. 13 is a tenth explanatory diagram in which the measured electrocardiographic potential time series is used for illustrating the certainty of the estimation result by the electromotive force estimation method of the embodiment.

FIG. 14 is an eleventh explanatory diagram in which the measured electrocardiographic potential time series is used for illustrating the certainty of the estimation result by the electromotive force estimation method of the embodiment.

FIG. 15 is a twelfth explanatory diagram in which the measured electrocardiographic potential time series is used for illustrating the certainty of the estimation result by the electromotive force estimation method of the embodiment.

FIG. 16 is a thirteenth explanatory diagram in which the measured electrocardiographic potential time series is used for illustrating the certainty of the estimation result by the electromotive force estimation method of the embodiment.

FIG. 17 is a fourteenth explanatory diagram in which the measured electrocardiographic potential time series is used for illustrating the certainty of the estimation result by the electromotive force estimation method of the embodiment.

FIG. 18 is a fifteenth explanatory diagram in which the measured electrocardiographic potential time series is used for illustrating the certainty of the estimation result by the electromotive force estimation method of the embodiment.

FIG. 19 is a sixteenth explanatory diagram in which the measured electrocardiographic potential time series is used for illustrating the certainty of the estimation result by the electromotive force estimation method of the embodiment.

FIG. 20 is a seventeenth explanatory diagram in which the measured electrocardiographic potential time series is used for illustrating the certainty of the estimation result by the electromotive force estimation method of the embodiment.

FIG. 21 is an eighteenth explanatory diagram in which the measured electrocardiographic potential time series is used for illustrating the certainty of the estimation result by the electromotive force estimation method of the embodiment.

FIG. 22 is a nineteenth explanatory diagram in which the measured electrocardiographic potential time series is used for illustrating the certainty of the estimation result by the electromotive force estimation method of the embodiment.

FIG. 23 is a twentieth explanatory diagram in which the measured electrocardiographic potential time series is used for illustrating the certainty of the estimation result by the electromotive force estimation method of the embodiment.

FIG. 24 is a twenty-first explanatory diagram in which the measured electrocardiographic potential time series is used for illustrating the certainty of the estimation result by the electromotive force estimation method of the embodiment.

FIG. 25 is a twenty-second explanatory diagram in which the measured electrocardiographic potential time series is used for illustrating the certainty of the estimation result by the electromotive force estimation method of the embodiment.

FIG. 26 is a twenty-third explanatory diagram in which the measured electrocardiographic potential time series is used for illustrating the certainty of the estimation result by the electromotive force estimation method of the embodiment.

FIG. 27 is a twenty-fourth explanatory diagram in which the measured electrocardiographic potential time series is used for illustrating the certainty of the estimation result by the electromotive force estimation method of the embodiment.

FIG. 28 is a twenty-fifth explanatory diagram in which the measured electrocardiographic potential time series is used for illustrating the certainty of the estimation result by the electromotive force estimation method of the embodiment.

FIG. 29 is a twenty-sixth explanatory diagram in which the measured electrocardiographic potential time series is used for illustrating the certainty of the estimation result by the electromotive force estimation method of the embodiment.

FIG. 30 is a twenty-seventh explanatory diagram in which the measured electrocardiographic potential time series is used for illustrating the certainty of the estimation result by the electromotive force estimation method of the embodiment.

FIG. 31 is a twenty-eighth explanatory diagram in which the measured electrocardiographic potential time series is used for illustrating the certainty of the estimation result by the electromotive force estimation method of the embodiment.

FIG. 32 is a twenty-ninth explanatory diagram in which the measured electrocardiographic potential time series is used for illustrating the certainty of the estimation result by the electromotive force estimation method of the embodiment.

FIG. 33 is a thirtieth explanatory diagram in which the measured electrocardiographic potential time series is used for illustrating the certainty of the estimation result by the electromotive force estimation method of the embodiment.

FIG. 34 is a thirty-first explanatory diagram in which the measured electrocardiographic potential time series is used for illustrating the certainty of the estimation result by the electromotive force estimation method of the embodiment.

FIG. 35 is a thirty-second explanatory diagram in which the measured electrocardiographic potential time series is used for illustrating the certainty of the estimation result by the electromotive force estimation method of the embodiment.

FIG. 36 is a thirty-third explanatory diagram in which the measured electrocardiographic potential time series is used for illustrating the certainty of the estimation result by the electromotive force estimation method of the embodiment.

FIG. 37 is a diagram illustrating an example of a configuration of an electromotive force estimation system 100 that executes the electromotive force estimation method of the embodiment.

FIG. 38 is an explanatory diagram illustrating an effect of matching processing in a modified example.

### DESCRIPTION OF EMBODIMENTS

Hereinafter, an electromotive force estimation method of an embodiment will be described with reference to the drawings. The “depolarization period” in the present disclosure is defined as QRS in an electrocardiogram. The depolarization period corresponds to the depolarization phase and spikes (Phase0 and Phase1) in the population of cardiomyocytes in the ventricle.

The “repolarization period” in the present disclosure is defined as T of the electrocardiogram. The repolarization period corresponds to the repolarization phase (Phase3) in the population of cardiomyocytes in the ventricle. The “refractory period” in the present disclosure is defined as the ST segment of the electrocardiogram. The refractory period corresponds to the plateau phase (Phase2) in the population of cardiomyocytes in the ventricle. The “resting period” in the present disclosure is defined as a period that is not any of the depolarization period, the refractory period, or the repolarization period. The resting period corresponds to the period of the static potential (Phase4) in the population of cardiomyocytes in the ventricle.

The spike (Phase1) in the depolarization period in the population of cardiomyocytes in the ventricle may be omitted from the depolarization period and instead included in the refractory period.

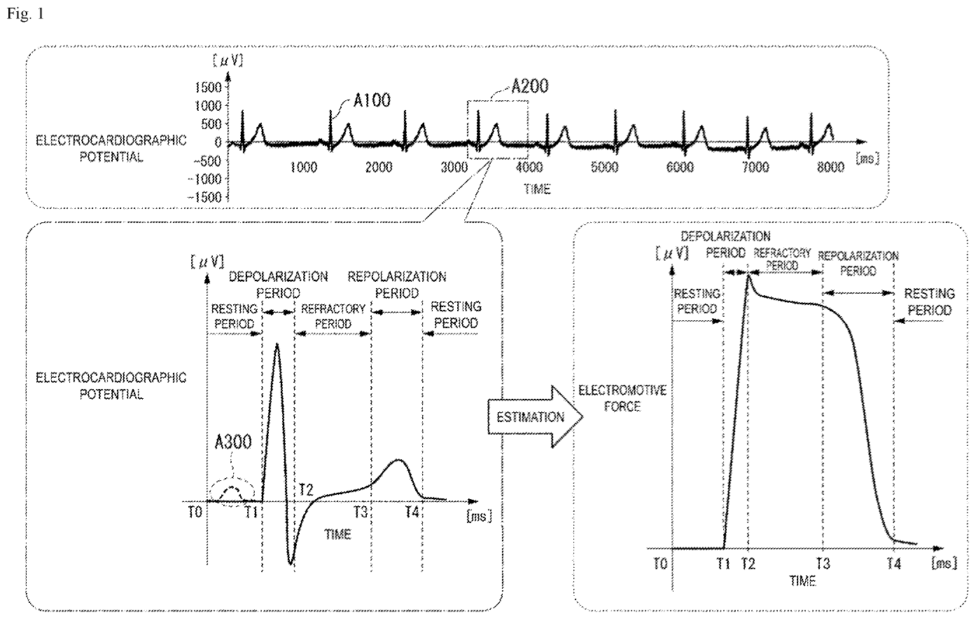

FIG. 1 is an explanatory diagram for explaining an outline of the electromotive force estimation method of the embodiment. The electromotive force estimation method is a method of estimating a time series of electromotive force (hereinafter referred to as “electromotive force time series”) in a period indicated by an electrocardiographic potential time series to be estimated based on a time series of electrocardiographic potentials (hereinafter referred to as “electrocardiographic potential time series”). The estimation target may be a person or an animal. Hereinafter, for simplicity of explanation, the electromotive force estimation method will be described using a case where the estimation target is a person as an example. In FIG. 1, a time series A100 is an example of the electrocardiographic potential time series. In FIG. 1, the unit of electrocardiographic potential is microvolt. In FIG. 1, the unit of time is millisecond.

In the electromotive force estimation method, first, an electrocardiographic potential time series is acquired. In the electromotive force estimation method, the acquired electrocardiographic potential time series is divided such that time series representing the electrocardiographic potential generated by one pulsation of the myocardium are separated from each other. One pulsation of the myocardium is a series of operations of the myocardium in a period from a time point when depolarization occurs in the myocardium to a time point when the heart enters a resting period through a depolarization period, a refractory period, and a repolarization period.

In FIG. 1, the time series A200 is a time series including a time series representing the electrocardiographic potential generated by one pulsation of the myocardium, and is an example of one time series after division (hereinafter referred to as “unit electrocardiographic potential time series”). The unit electrocardiographic potential time series is, for example, an electrocardiographic potential time series in a period in which the start is a time point of the start of the P wave and the end is a time point immediately before the start of the next P wave. For example, in a case where it is difficult to detect the P wave, the unit electrocardiographic potential time series may be an electrocardiographic potential time series in a period satisfying the condition of from the base line to return to the base line through the R wave T wave.

The unit electrocardiographic potential time series includes a depolarization electrocardiographic potential time series, a refractory electrocardiographic potential time series, a repolarization electrocardiographic potential time series, and a resting electrocardiographic potential time series. The depolarization electrocardiographic potential time series is a time series indicating the electrocardiographic potential generated in the depolarization period. The refractory electrocardiographic potential time series is a time series indicating the electrocardiographic potential generated in the refractory period. The repolarization electrocardiographic potential time series is a time series indicating the electrocardiographic potential generated in the repolarization period. The resting electrocardiographic potential time series is a time series indicating the electrocardiographic potential generated in the resting period. That is, the resting electrocardiographic potential time series is a set of data that does not belong to any of the depolarization electrocardiographic potential time series, the refractory electrocardiographic potential time series, and the repolarization electrocardiographic potential time series in the pieces of data of the unit electrocardiographic potential time series.

In FIG. 1, a time series in a period from time T1 to time T2 is an example of a depolarization electrocardiographic potential time series. In FIG. 1, a time series in a period from time T2 to time T3 is an example of a refractory electrocardiographic potential time series. In FIG. 1, a time series in a period from time T3 to time T4 is an example of a repolarization electrocardiographic potential time series. In FIG. 1, a time series in a period from time T0 to time T1 and a time series after time T4 are examples of the resting electrocardiographic potential time series.

In FIG. 1, the unit electrocardiographic potential time series is substantially constant from time T0 to time T1. In FIG. 1, a time series A300 in a period from time T0 to time T1 is a time series indicating the P wave.

In the electromotive force estimation method, a time series of electromotive force in a period indicated by each unit electrocardiographic potential time series (hereinafter referred to as “unit electromotive force time series”) is estimated for each unit electrocardiographic potential time series. Specifically, first, pieces of data indicated by the unit electrocardiographic potential time series are classified into data belonging to the depolarization electrocardiographic potential time series, data belonging to the refractory electrocardiographic potential time series, data belonging to the repolarization electrocardiographic potential time series, and data belonging to the resting electrocardiographic potential time series.

Hereinafter, the data belonging to the depolarization electrocardiographic potential time series is referred to as “depolarization electrocardiographic potential time series data”. Hereinafter, the data belonging to the refractory electrocardiographic potential time series is referred to as “refractory electrocardiographic potential time series data”. Hereinafter, the data belonging to the repolarization electrocardiographic potential time series is referred to as “repolarization electrocardiographic potential time series data”. Hereinafter, the data belonging to the resting electrocardiographic potential time series is referred to as “resting electrocardiographic potential time series data”.

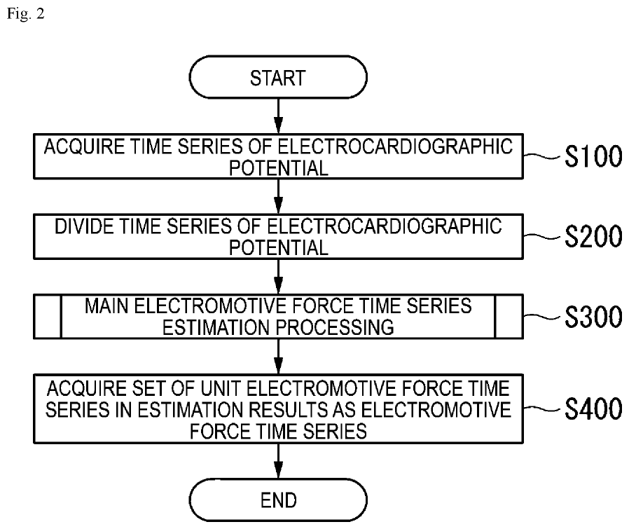

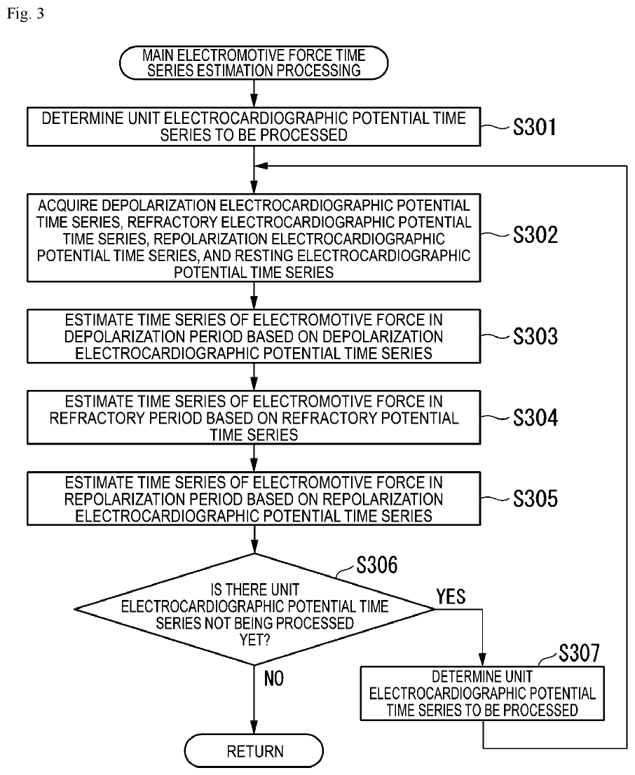

Next, the electromotive force time series is estimated according to a rule corresponding to each period of the depolarization period, the refractory period, and the repolarization period. Hereinafter, the electromotive force estimation method will be described together with a description of a specific estimation method of estimating the time series of the electromotive force for each unit electrocardiographic potential time series with reference to FIGS. 2 and 3.

FIG. 2 is a flowchart illustrating an example of a flow of processing executed in the electromotive force estimation method of the embodiment. The electromotive force estimation method is executed by a computer, for example.

The electrocardiographic potential time series is acquired (step S100). Next, the acquired electrocardiographic potential time series is divided for each unit electrocardiographic potential time series (step S200). The processing in step S200 is processing of dividing the electrocardiographic potential time series by the unit electrocardiographic potential time series, that is processing of generating the unit electrocardiographic potential time series.

Next, main electromotive force time series estimation processing is executed (step S300). The main electromotive force time series estimation processing is processing of estimating a unit electromotive force time series in a period indicated by each unit electrocardiographic potential time series for all the unit electrocardiographic potential time series generated in step S200.

Specifically, the main electromotive force time series estimation processing is processing of executing sub electromotive force time series estimation processing for all the unit electrocardiographic potential time series. The sub electromotive force time series estimation processing is processing executed for one unit electrocardiographic potential time series, and is processing of estimating a unit electromotive force time series in a period indicated by the unit electrocardiographic potential time series to be executed. Hereinafter, the unit electrocardiographic potential time series to be subjected to the sub electromotive force time series estimation processing is referred to as a “target unit electrocardiographic potential time series”.

FIG. 3 is a flowchart illustrating an example of a flow of main unit electromotive force time series estimation processing of the embodiment. According to a predetermined rule, one of the unit electrocardiographic potential time series generated in step S200 is determined as the target unit electrocardiographic potential time series (step S301). The predetermined rule is, for example, a rule of determining the unit electrocardiographic potential time series of the earliest period among the unit electrocardiographic potential time series acquired in step S200 as the target unit electrocardiographic potential time series.

Processing from step S302 to step S305 described below is an example of the sub electromotive force time series estimation processing. After step S301, pieces of data of the target unit electrocardiographic potential time series are classified into any one of the depolarization electrocardiographic potential time series data, the refractory electrocardiographic potential time series data, the repolarization electrocardiographic potential time series data, or the resting electrocardiographic potential time series data (step S302).

A set of data classified into the depolarization electrocardiographic potential time series data by the processing in step S302 is the depolarization electrocardiographic potential time series. A set of data classified into the refractory electrocardiographic potential time series data by the processing in step S302 is the refractory electrocardiographic potential time series. A set of data classified into the repolarization electrocardiographic potential time series data by the processing in step S302 is the repolarization electrocardiographic potential time series. A set of data classified into the resting electrocardiographic potential time series data by the processing in step S302 is the resting electrocardiographic potential time series.

As described above, the processing in step S302 is processing of acquiring the depolarization electrocardiographic potential time series, the refractory electrocardiographic potential time series, the repolarization electrocardiographic potential time series, and the resting electrocardiographic potential time series.

The target unit electrocardiographic potential time series classification method may be any method as long as pieces of data of the target unit electrocardiographic potential time series can be classified into any one of the depolarization electrocardiographic potential time series data, the refractory electrocardiographic potential time series data, the repolarization electrocardiographic potential time series data, or the resting electrocardiographic potential time series data. The target unit electrocardiographic potential time series classification method is a method of classifying pieces of data of the target unit electrocardiographic potential time series into any one of the depolarization electrocardiographic potential time series data, the refractory electrocardiographic potential time series data, the repolarization electrocardiographic potential time series data, or the resting electrocardiographic potential time series data.

The method of determining the depolarization electrocardiographic potential time series data in the target unit electrocardiographic potential time series classification method is, for example, a method of determining whether or not the time point indicated by the data is the depolarization period after determining the start time point of the depolarization period and the end time point of the depolarization period. The start time point of the period means the time point of the start of the period. The end time point of the period means the time point of the end of the period.

The start time point of the depolarization period is, for example, a time point when the condition that the change amount per unit time of the electrocardiographic potential indicated by the target unit electrocardiographic potential time series is equal to or more than a predetermined amount is first satisfied. The start time point of the depolarization period may be, for example, a condition that the absolute value of the change amount of the electrocardiographic potential per unit time of 5 msec is 40 μvolt or more in the case of a 200 Hz sampling frequency. The threshold value is not limited to this value, and may be a value adjusted by measurement conditions such as the magnitude of the electrocardiographic potential, the skin properties, the bioelectrode, and the gain of the measuring instrument. In addition, the start time point of the depolarization period may be, for example, a time point calculated from an inflection point obtained by differentiation of the electrocardiographic potential or the like.

The end time point of the depolarization period is, for example, a time point after the start time point of the depolarization, and is a time point when the change amount of the electrocardiographic potential indicated by the target unit electrocardiographic potential time series falls below the predetermined threshold value for the first time after the sign of the slope in the positive direction on the time axis of the electrocardiographic potential indicated by the target unit electrocardiographic potential time series is inverted at least once. The end time point of the depolarization period may be, for example, a condition that the absolute value of the change amount of the electrocardiographic potential per unit time of 5 msec is 30 μvolt or less. The threshold value is not limited to this value, and may be a value adjusted by measurement conditions such as the magnitude of the electrocardiographic potential, the skin properties, the noise, the bioelectrode, and the gain of the measuring instrument. In addition, the end time point of the depolarization period may be, for example, a time point calculated from an inflection point obtained by differentiation of the electrocardiographic potential or the like.

The method of determining the refractory electrocardiographic potential time series data in the target unit electrocardiographic potential time series classification method is, for example, a method of determining whether or not the time point indicated by the data is the refractory period after determining the start time point of the refractory period and the end time point of the refractory period.

The start time point of the refractory period is a time point immediately after the end time point of the depolarization period. The end time point of the refractory period is, for example, a time point when the condition that the change amount in the negative direction per unit time of the electrocardiographic potential indicated by the target unit electrocardiographic potential time series is equal to or more than a predetermined amount is first satisfied after the start time point of the refractory period. The end time point of the refractory period may be a time point when, for example, the condition that the absolute value of the change amount of the electrocardiographic potential is 10 μvolt or more. The threshold value is not limited to this value, and may be a value adjusted by measurement conditions such as the magnitude of the electrocardiographic potential, the skin properties, the noise, the bioelectrode, and the gain of the measuring instrument. In addition, the end time point of the refractory period may be, for example, a time point calculated from an inflection point obtained by differentiation of the electrocardiographic potential or the like.

The method of determining the repolarization electrocardiographic potential time series data in the target unit electrocardiographic potential time series classification method is, for example, a method of determining whether or not the time point indicated by the data is the repolarization period after determining the start time point of the repolarization period and the end time point of the repolarization period.

The start time point of the repolarization period is a time point immediately after the end time point of the refractory period. The end time point of the repolarization period is, for example, a time point when the condition that the absolute value of the change amount per unit time of the electrocardiographic potential indicated by the target unit electrocardiographic potential time series is less than a predetermined amount is first satisfied after the start time point of the repolarization period. At the end time point of the repolarization period, the absolute value of the change amount of the electrocardiographic potential may be less than 7 μvolt. The threshold value is not limited to this value, and may be a value adjusted by measurement conditions such as the magnitude of the electrocardiographic potential, the skin properties, the noise, the bioelectrode, and the gain of the measuring instrument. In addition, the end time point of the repolarization period may be, for example, a time point calculated from an inflection point obtained by differentiation of the electrocardiographic potential or the like.

After step S302, a time series of the electromotive force in the depolarization period (hereinafter referred to as “depolarization electromotive force time series”) is estimated based on the depolarization electrocardiographic potential time series acquired in step S302 (step S303). The depolarization electromotive force time series is data having a depolarized electromotive force estimation value for each time point of the depolarization electrocardiographic potential time series. The depolarized electromotive force estimation value at each time point is an estimation value of the electromotive force at each time point in the depolarization period.

Specifically, the depolarized electromotive force estimation value is a value obtained by adding the sum of past depolarization difference potentials indicated by the depolarization electrocardiographic potential time series acquired in step S302 to the electrocardiographic potential at the start time point of the depolarization period indicated by the depolarization electrocardiographic potential time series acquired in step S302.

The depolarization difference potential is the absolute value of the difference in electrocardiographic potential between two adjacent time points in the depolarization period. The past depolarization difference potential does not include the depolarization difference potential in different unit electrocardiographic potential time series. The past depolarization difference potential is a past depolarization difference potential after the start time point of the depolarization period.

The processing of estimating the depolarization electromotive force time series in this manner is processing of estimating the electromotive force at each time point for each time point of the depolarization period. Specifically, the processing of estimating the depolarization electromotive force time series is processing of acquiring, as the electromotive force, a value obtained by adding the sum of the absolute values of the differences between adjacent electrocardiographic potentials from the start time point of the depolarization period to the time point corresponding to the electromotive force to be estimated to the electrocardiographic potential at the start time point of the depolarization period.

After step S303, a time series of the electromotive force in the refractory period (hereinafter referred to as “refractory electromotive force time series”) is estimated based on the refractory electrocardiographic potential time series acquired in step S302 and the end time point depolarized electromotive force estimation value acquired in step S303 (step S304). The end time point depolarized electromotive force estimation value is a depolarized electromotive force estimation value at the end time point of the depolarization period.

The refractory electromotive force time series is data having a refractory electromotive force estimation value for each time point of the refractory electrocardiographic potential time series. The refractory electromotive force estimation value at each time point is an estimation value of the electromotive force at each time point in the refractory period. Specifically, the refractory electromotive force estimation value at each time point is a value obtained by subtracting a value related to the electrocardiographic potential at a corresponding time point of the refractory electrocardiographic potential time series from the value of the end time point depolarized electromotive force estimation value.

The value related to the electrocardiographic potential of the refractory electrocardiographic potential time series used for the subtraction is, for example, a value obtained by subtracting the electrocardiographic potential at the corresponding time point of the refractory electrocardiographic potential time series from the value of the end time point depolarized electromotive force estimation value. The value related to the electrocardiographic potential of the refractory electrocardiographic potential time series used for the subtraction may be a measurement value of the electrocardiographic potential or an absolute value of the electrocardiographic potential. Depending on the pattern of the electrocardiographic potential, unnatural deformation such as inversion, folding, or bending may occur depending on the positional relationship with the baseline. In such a case, the value related to the electrocardiographic potential of the refractory electrocardiographic potential time series used for the subtraction may be a value obtained by subtraction after applying the deformation processing to the refractory period electrocardiographic potential. In this modification, adjustment such as adding a gain (multiple) or a bias (constant) or using accumulation may be performed. The corresponding time point in the definition of the refractory electromotive force estimation value is a time point indicated by the refractory electrocardiographic potential time series data, and is the same time point as the time point indicated by the refractory electromotive force time series data indicating the refractory electromotive force estimation value.

The processing of estimating the refractory electromotive force time series in this manner is processing of estimating the electromotive force at each time point for each time point of the refractory period. Specifically, it is processing of acquiring, as the electromotive force, a value obtained by subtracting a value related to the electrocardiographic potential at a corresponding time point in the refractory period from the estimation value of the electromotive force at the end time point of the depolarization period.

The processing of estimating the refractory electromotive force time series is, for example, processing of estimating the electromotive force at each time point for each time point of the refractory period. Specifically, it is processing of acquiring, as the electromotive force, a value obtained by subtracting the electrocardiographic potential at a corresponding time point in the refractory period from the estimation value of the electromotive force at the end time point of the depolarization period. The processing of estimating the refractory electromotive force time series may be, for example, processing of acquiring, as the electromotive force, a value obtained by subtracting the deformed electrocardiographic potential at the corresponding time point in the refractory period from the estimation value of the electromotive force at the end time point of the depolarization period.

Hereinafter, for the sake of simplicity of explanation, the electromotive force estimation method will be described by taking, as an example, a case where the value related to the electrocardiographic potential of the refractory electrocardiographic potential time series used for the subtraction is a value obtained by subtracting the electrocardiographic potential at the corresponding time point of the refractory electrocardiographic potential time series from the value of the end time point depolarized electromotive force estimation value. The processing of estimating the refractory electromotive force time series is, and the processing of estimating the refractory electromotive force time series is, for example, processing of estimating the electromotive force at each time point for each time point of the refractory period.

After step S304, a time series of the electromotive force in the repolarization period (hereinafter referred to as “repolarization electromotive force time series”) is estimated based on the repolarization electrocardiographic potential time series acquired in step S302 and the end time point refractory electromotive force estimation value acquired in step S304 (step S305). The end time point refractory electromotive force estimation value is a refractory electromotive force estimation value at the end time point of the refractory period. The end time point refractory electromotive force estimation value coincides with the repolarization electromotive force estimation value at the start time point of the repolarization period.

The repolarization electromotive force time series is data having a repolarization electromotive force estimation value for each time point of the repolarization electrocardiographic potential time series. The repolarization electromotive force estimation value at each time point is an estimation value of the electromotive force at each time point in the repolarization period. Specifically, the repolarization electromotive force estimation value at each time point is a value obtained by subtracting the sum of past repolarization difference potentials indicated by the repolarization electrocardiographic potential time series acquired in step S302 from the end time point refractory electromotive force estimation value.

The repolarization difference potential is a value based on the difference in electrocardiographic potential between two adjacent time points in the repolarization period. The repolarization difference potential is, for example, the absolute value of the difference in electrocardiographic potential between two adjacent time points in the repolarization period. The past repolarization difference potential does not include the repolarization difference potential in different unit electrocardiographic potential time series. Accordingly, the past repolarization difference potential is the past repolarization difference potential after the end time point of the refractory period. The repolarization period electrocardiographic potential used for this subtraction may be, for example, a value obtained by performing adjustment such as adding a bias or a gain or using accumulation to the absolute value of the electrocardiographic potential, instead of the absolute value of the electrocardiographic potential in a case where the electrocardiographic potential crosses a baseline. Hereinafter, for the sake of simplicity of explanation, the electromotive force estimation method will be described by taking, as an example, a case where the repolarization difference potential is the absolute value of the difference in electrocardiographic potential between two adjacent time points in the repolarization period.

The processing of estimating the repolarization electromotive force time series in this manner is processing of estimating the electromotive force at each time point for each time point of the repolarization period. Specifically, it is processing of acquiring, as the electromotive force, a value obtained by subtracting the sum of the absolute values of the differences between adjacent electrocardiographic potentials from the end time point of the refractory period in the repolarization electrocardiographic potential time series to the time point corresponding to the estimated electromotive force from the estimation value of the electromotive force at the end time point of the refractory period.

A set of the depolarization electromotive force time series, the refractory electromotive force time series, and the repolarization electromotive force time series estimated by one execution of the series of processing from step S302 to step S305 is an estimated unit electromotive force time series in a period indicated by the target unit electrocardiographic potential time series.

After step S305, it is determined whether or not the unit electromotive force time series has been estimated for all the unit electrocardiographic potential time series generated in step S200 (step S306). That is, it is determined whether there is no unestimated unit electrocardiographic potential time series. The unestimated unit electrocardiographic potential time series is a unit electrocardiographic potential time series on which the sub electromotive force time series estimation processing is not executed.

When there is an unestimated unit electrocardiographic potential time series (step S306: YES), one of the unestimated unit electrocardiographic potential time series is determined as the target unit electrocardiographic potential time series according to a predetermined rule (step S307). The predetermined rule is, for example, a rule of determining the unestimated unit electrocardiographic potential time series of the earliest period among the unestimated unit electrocardiographic potential time series as the target unit electrocardiographic potential time series. After step S307, the processing returns to step S302. On the other hand, when there is no unestimated unit electrocardiographic potential time series (step S306: NO), the processing ends.

FIG. 1 will be referred to again for explanation. A set of each unit electromotive force time series estimated by the processing in step S300 is acquired as an estimation result of the electromotive force time series in the period indicated by the electrocardiographic potential time series acquired in step S100 (step S400).

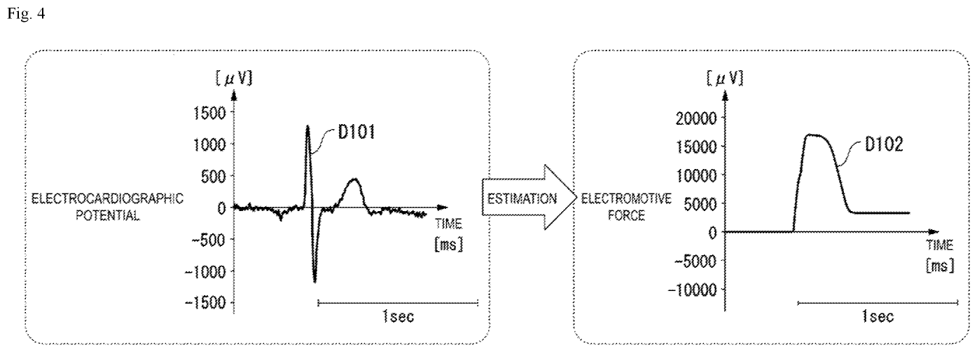

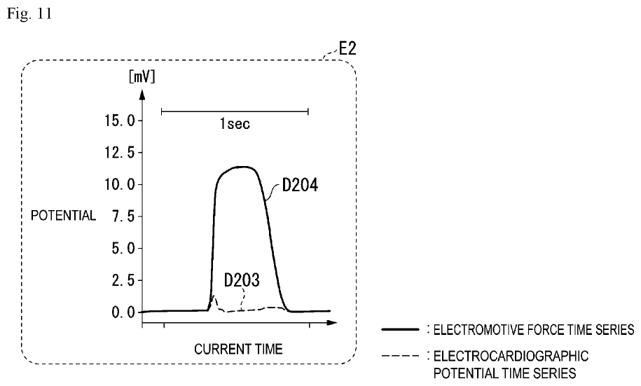

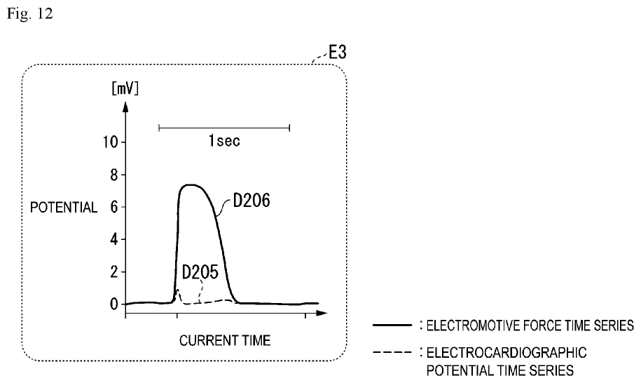

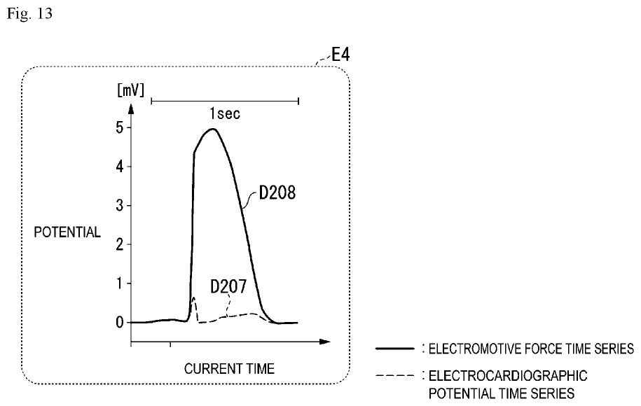

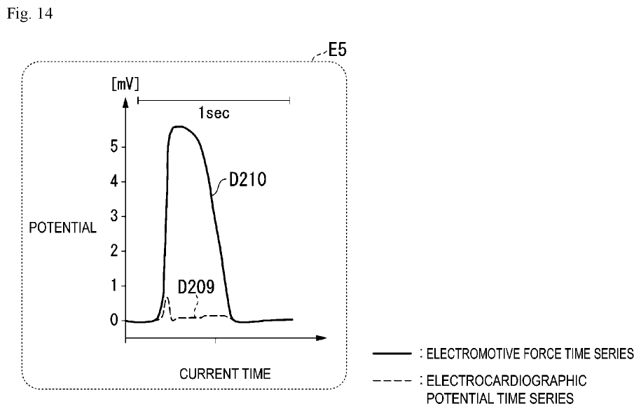

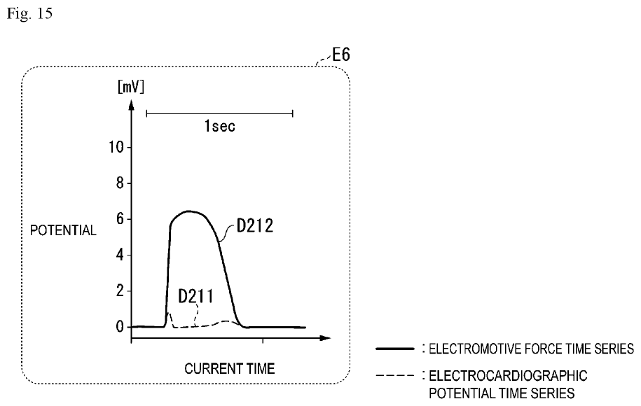

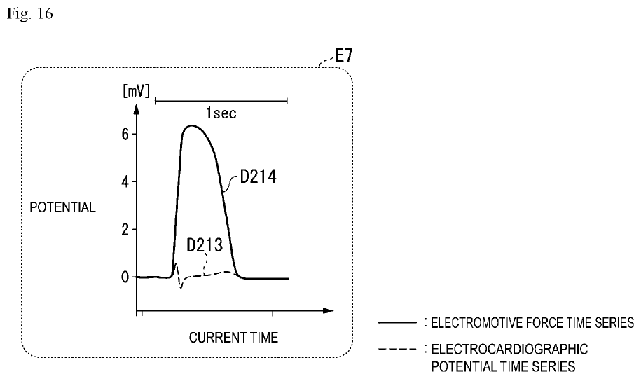

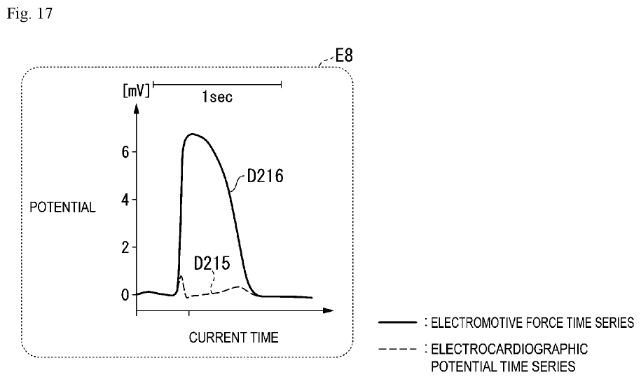

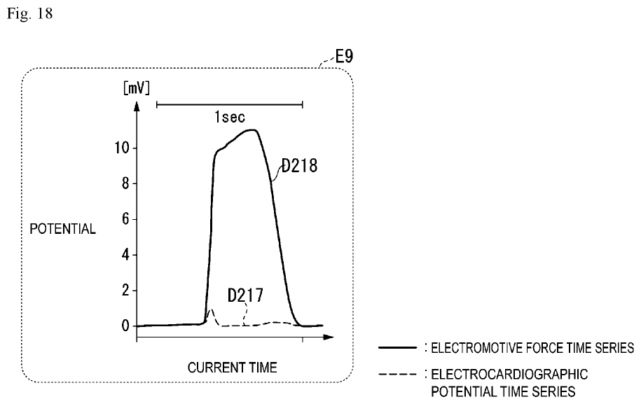

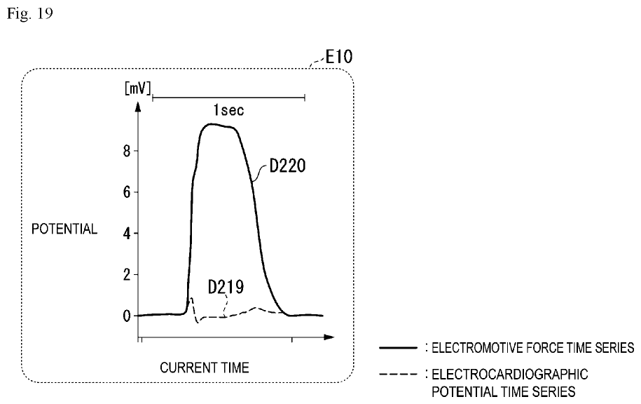

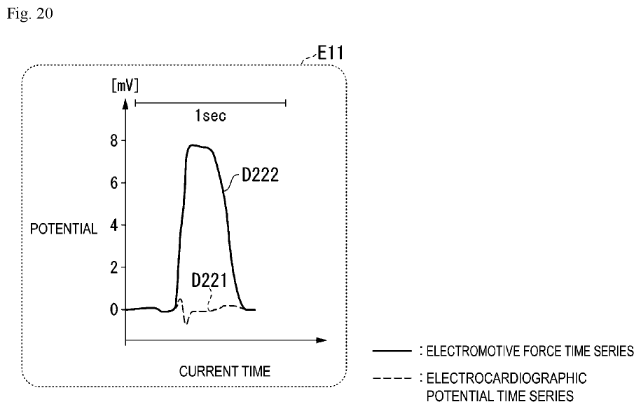

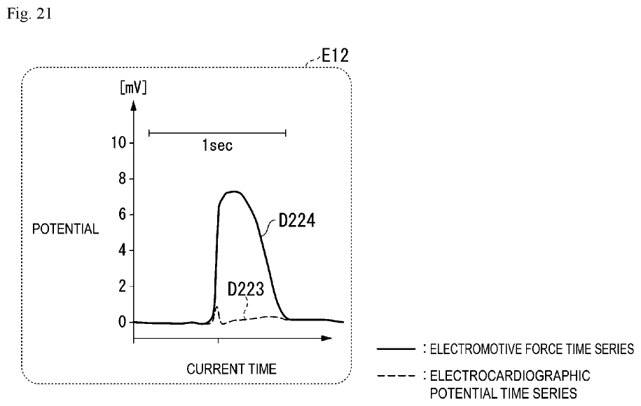

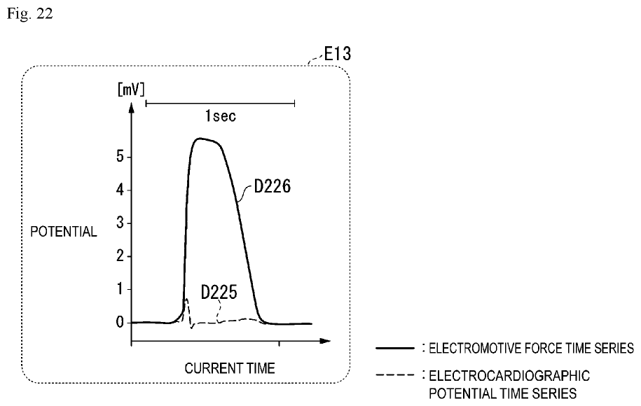

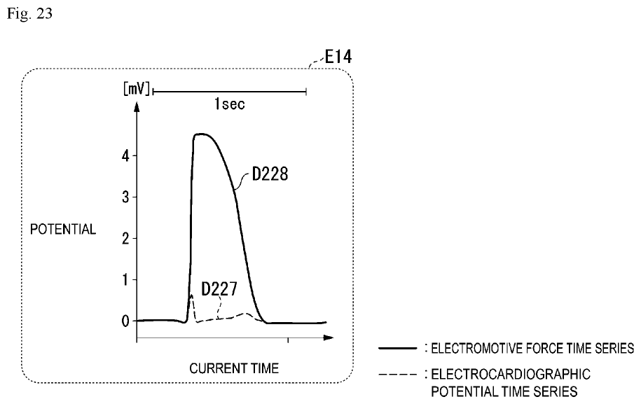

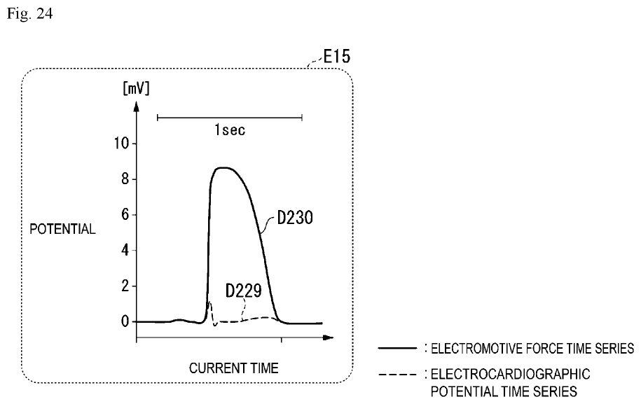

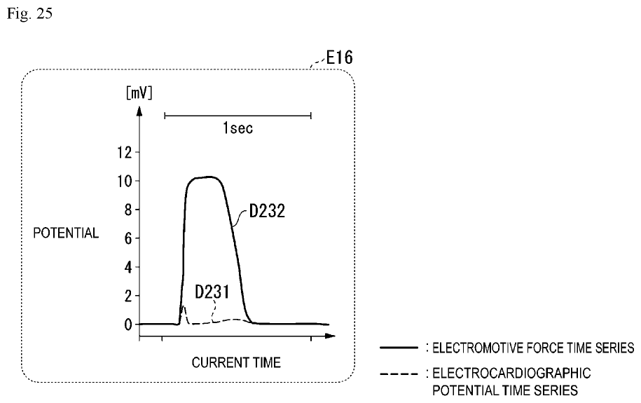

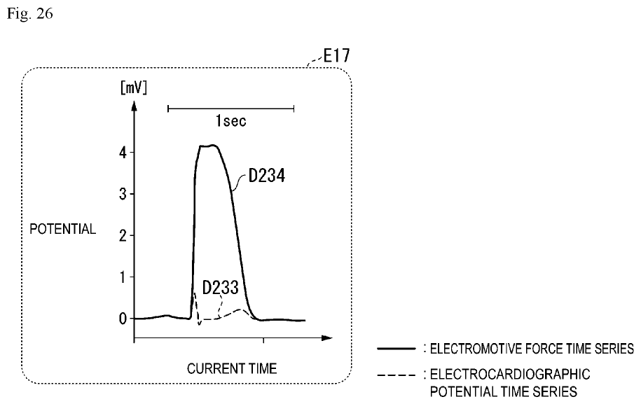

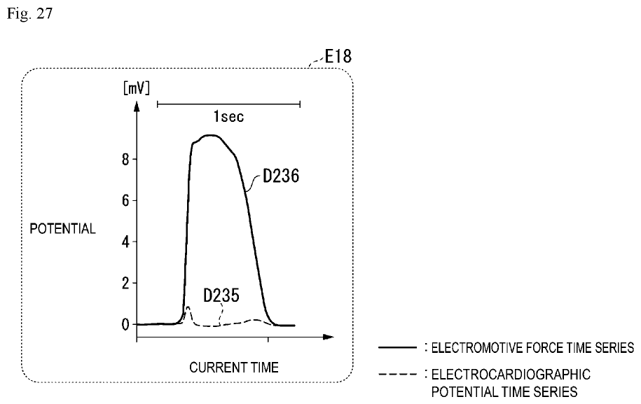

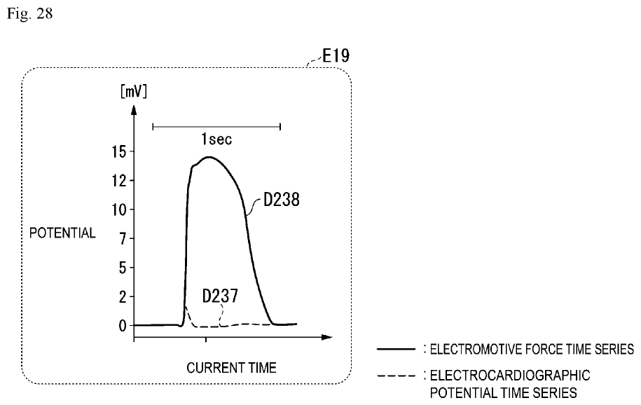

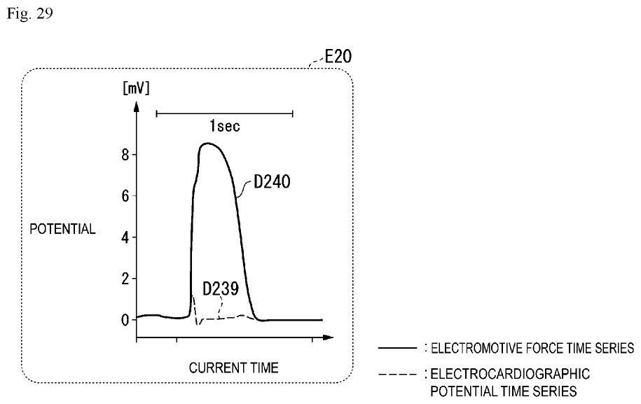

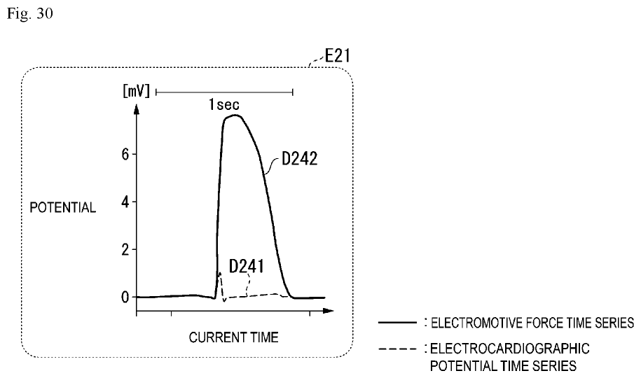

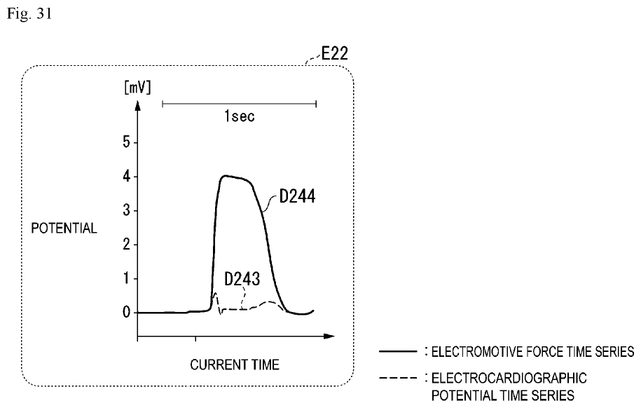

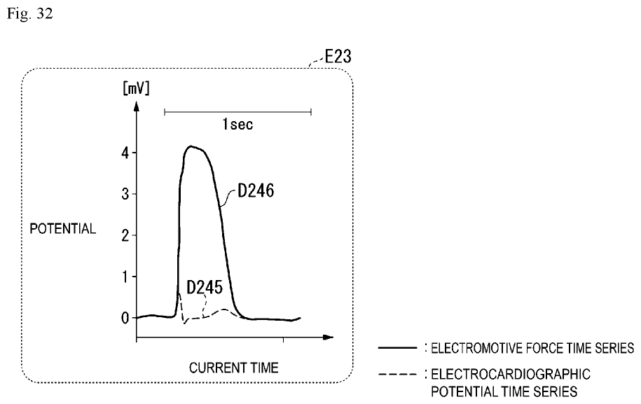

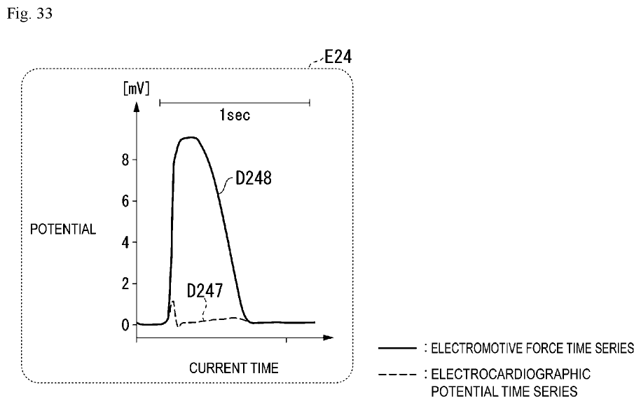

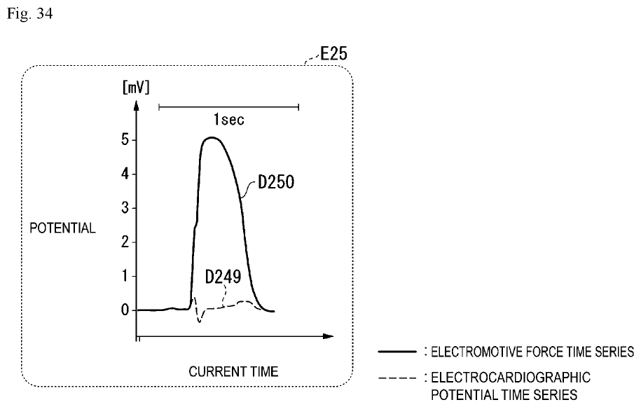

Hereinafter, a verification result of the certainty of the estimation result by the electromotive force estimation method of the embodiment will be described by using the measured electrocardiographic potential time series with reference to FIGS. 4 to 34. In the description of FIGS. 4 to 34, for simplicity of explanation, the electromotive force estimation method will be described by taking a case where the electrocardiographic potential time series is a time series of the electrocardiographic potential generated by one pulsation of the myocardium as an example. The electromotive force series of the estimation result is one unit electrocardiographic potential time series.

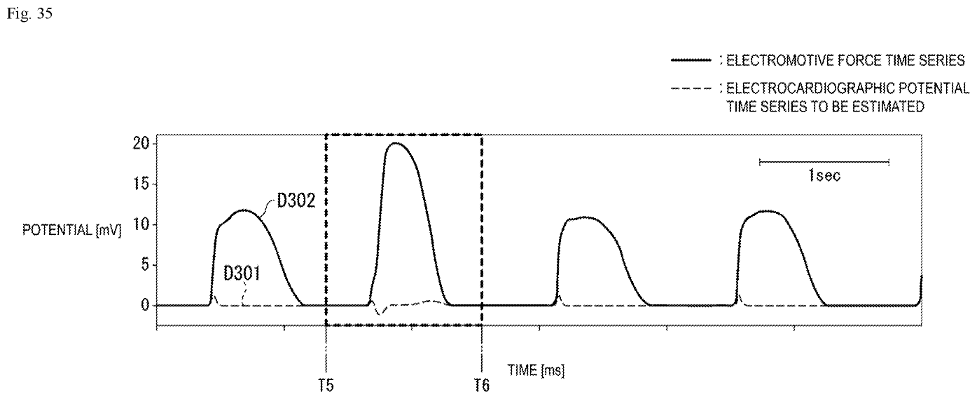

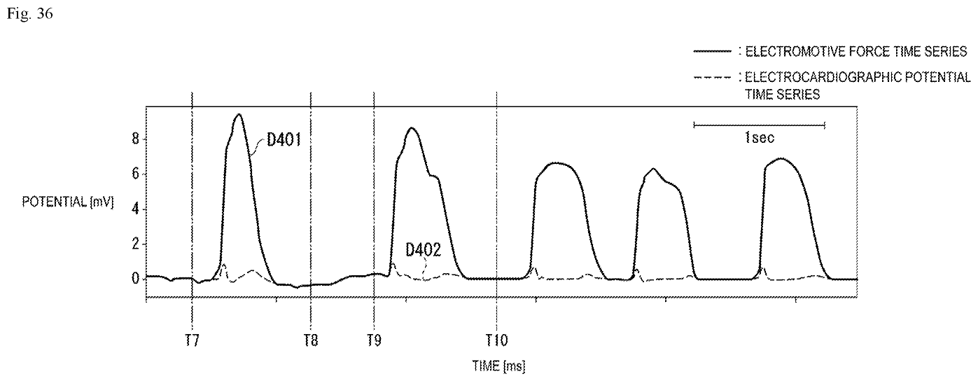

In addition, with reference to FIGS. 35 and 36, an example of an estimation result of the electrocardiographic potential time series by the electromotive force estimation method of the embodiment will be described by taking, as an example, a case where the electrocardiographic potential time series is the time series of the electrocardiographic potential generated by a plurality of pulsations of the myocardium.

The measured electrocardiographic potential time series in the description of FIGS. 4 to 36 is data published in PhysioNet (https://physionet.org/about/database/#ecg).

FIG. 4 is a first explanatory diagram in which the measured electrocardiographic potential time series is used for illustrating the certainty of the estimation result by the electromotive force estimation method of the embodiment. More specifically, FIG. 4 illustrates an example of the electrocardiographic potential time series of a person whose myocardium movement is normal and before an exercise stress test is performed, and an estimation result estimated by the electromotive force estimation method using the electrocardiographic potential time series.

A time series D101 in FIG. 4 indicates a measured electrocardiographic potential time series. A time series D102 in FIG. 4 indicates an electromotive force time series estimated by the electromotive force estimation method based on the time series D101. The time series D102 of the estimation result has a feature similar to a feature that is medically recognized as a feature of the electromotive force time series before the exercise load test of a person whose myocardium movement is normal. Specifically, the time series D102 of the estimation result has a feature of the action potential of the myocardium having a plateau due to persistence of the depolarization potential, and has a feature of the waveform of the myocardium observed by monophasic myocardial action potential recording or the like (see NPLs 1 and 4). FIG. 4 indicates that the electromotive force estimation method of the embodiment is a method of estimating the electromotive force time series before the exercise load test of a person whose myocardium movement is normal with high accuracy.

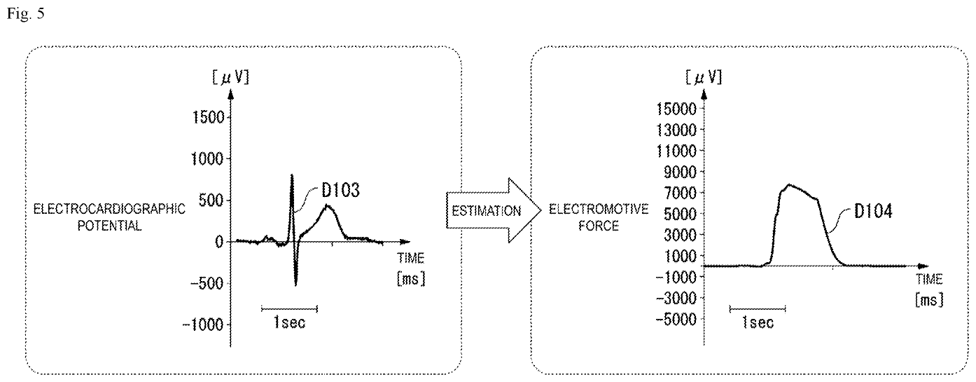

FIG. 5 is a second explanatory diagram in which the measured electrocardiographic potential time series is used for illustrating the certainty of the estimation result by the electromotive force estimation method of the embodiment. More specifically, FIG. 5 illustrates an example of the electrocardiographic potential time series of a person exhibiting transient ischemia in the myocardium and an estimation result estimated by the electromotive force estimation method using the electrocardiographic potential time series.

A time series D103 in FIG. 5 indicates a measured electrocardiographic potential time series. A time series D104 in FIG. 5 indicates an electromotive force time series estimated by the electromotive force estimation method based on the time series D103. The time series D104 of the estimation result has a feature similar to a feature that is medically recognized as a feature of the electromotive force time series at the time of myocardial ischemia. Specifically, it has a feature of delaying depolarization, changes such as rising or falling of a plateau, and bending, and prolongation of an action potential duration. FIG. 5 indicates that the electromotive force estimation method is a method of estimating the electromotive force time series at the time of myocardial ischemia with high accuracy.

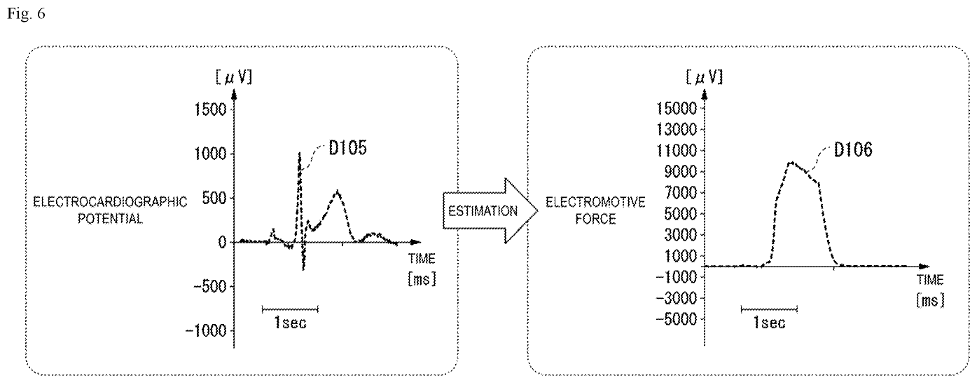

FIG. 6 is a third explanatory diagram in which the measured electrocardiographic potential time series is used for illustrating the certainty of the estimation result by the electromotive force estimation method of the embodiment. More specifically, FIG. 6 illustrates an example of the electrocardiographic potential time series at the time of myocardial ischemia and an estimation result estimated by the electromotive force estimation method using the electrocardiographic potential time series.

A time series D105 in FIG. 6 indicates a measured electrocardiographic potential time series. A time series D106 in FIG. 6 indicates an electromotive force time series estimated by the electromotive force estimation method based on the time series D105. The time series D106 of the estimation result has a feature similar to a feature that is medically recognized as a feature of the electromotive force time series at the time of myocardial ischemia. Specifically, it has a feature of delaying depolarization, changes such as rising or falling of a plateau, and bending, and prolongation of an action potential duration. FIG. 6 indicates that the electromotive force estimation method is a method of estimating the electromotive force time series at the time of myocardial ischemia with high accuracy.

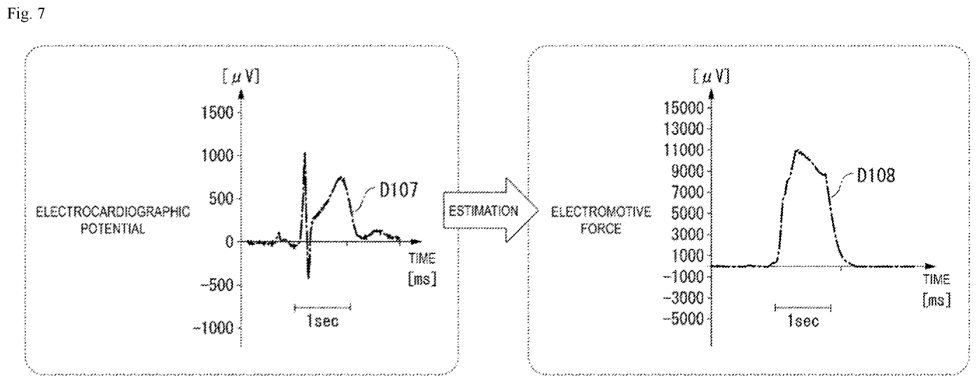

FIG. 7 is a fourth explanatory diagram in which the measured electrocardiographic potential time series is used for illustrating the certainty of the estimation result by the electromotive force estimation method of the embodiment. More specifically, FIG. 7 illustrates an example of the electrocardiographic potential time series at the time of myocardial ischemia and an estimation result estimated by the electromotive force estimation method using the electrocardiographic potential time series.

A time series D107 in FIG. 7 indicates a measured electrocardiographic potential time series. A time series D108 in FIG. 7 indicates an electromotive force time series estimated by the electromotive force estimation method based on the time series D107. The time series D108 of the estimation result has a feature similar to a feature that is medically recognized as a feature of the electromotive force time series at the time of myocardial ischemia. Specifically, it has a feature of delaying depolarization, changes such as rising or falling of a plateau, and bending, and prolongation of an action potential duration. FIG. 7 indicates that the electromotive force estimation method is a method of estimating the electromotive force time series at the time of myocardial ischemia with high accuracy.

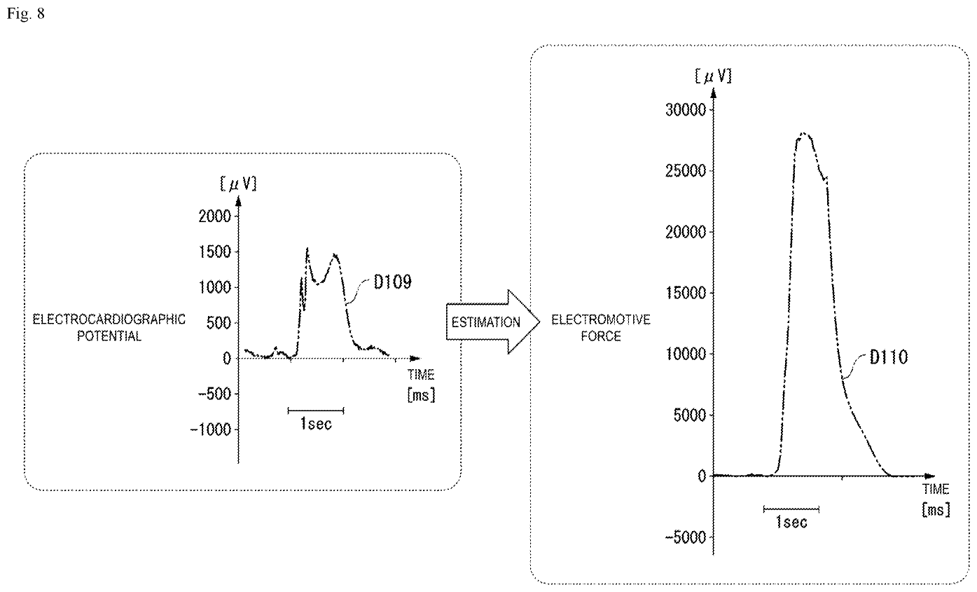

FIG. 8 is a fifth explanatory diagram in which the measured electrocardiographic potential time series is used for illustrating the certainty of the estimation result by the electromotive force estimation method of the embodiment. More specifically, FIG. 8 illustrates an example of the electrocardiographic potential time series at the time of myocardial ischemia and an estimation result estimated by the electromotive force estimation method using the electrocardiographic potential time series.

A time series D109 in FIG. 8 indicates a measured electrocardiographic potential time series. A time series D110 in FIG. 8 indicates an electromotive force time series estimated by the electromotive force estimation method based on the time series D109. The time series D110 of the estimation result has a feature similar to a feature that is medically recognized as a feature of the electromotive force time series at the time of myocardial ischemia. Specifically, it has a feature of delaying depolarization, changes such as rising or falling of a plateau, and bending, and prolongation of an action potential duration. FIG. 8 indicates that the electromotive force estimation method is a method of estimating the electromotive force time series at the time of myocardial ischemia with high accuracy.

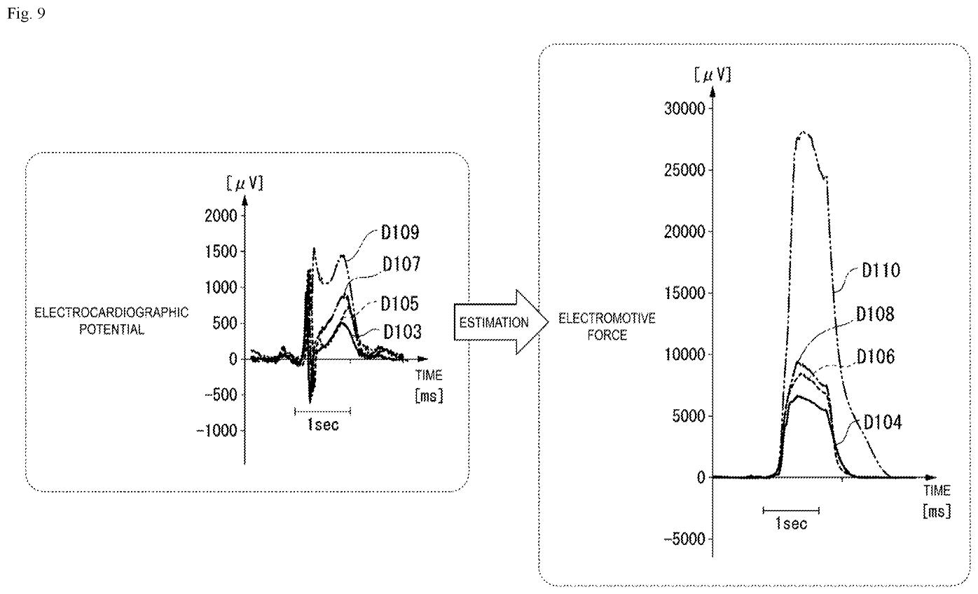

FIG. 9 is a sixth explanatory diagram in which the measured electrocardiographic potential time series is used for illustrating the certainty of the estimation result by the electromotive force estimation method of the embodiment. More specifically, FIG. 9 illustrates a result of superimposing and displaying the time series illustrated in FIGS. 5, 6, 7, and 8.

In FIG. 9, the vertical axis represents the potential and the horizontal axis represents time. The potentials indicated by the vertical axis indicate the electrocardiographic potential for the time series D103, D105, D107, and D109, and indicate the cardiac electromotive force for the time series D104, D106, D108, and D110.

FIG. 9 is a diagram illustrating that there is a difference reflecting a difference in electrocardiographic potential time series in each shape indicated by two electromotive force time series estimated by the electromotive force estimation method. Specifically, FIG. 9 indicates that there is a potential difference in the refractory period between electromotive force time series, and there is also a potential difference in the refractory period in electrocardiographic potential time series. In addition, regarding the difference in the refractory period in FIG. 9, the difference between the electromotive force time series is larger than the difference between the electrocardiographic potential time series. Thus, FIG. 9 indicates that a difference that amplifies the difference between the electrocardiographic potential time series appears as the difference between the electromotive force time series.

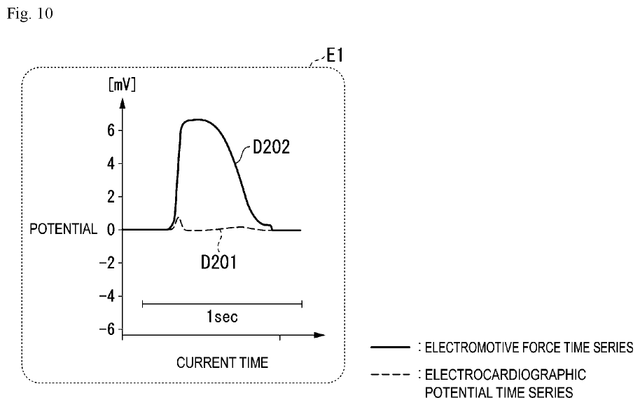

With reference to FIGS. 10 to 34, the certainty of the estimation result by the electromotive force estimation method of the embodiment will be described. FIGS. 10 to 34 are seventh to thirty-first explanatory diagrams in which the measured electrocardiographic potential time series is used for illustrating the certainty of the estimation result by the electromotive force estimation method of the embodiment. More specifically, each of the results in FIGS. 10 to 34 is an example illustrating the electrocardiographic potential time series of a person whose myocardium movement is normal and the estimation result estimated by the electromotive force estimation method using the electrocardiographic potential time series.

FIGS. 10 to 34 indicate results E1 to E25. The results E1 to E25 each indicates the measured electrocardiographic potential time series and the electromotive force time series of the estimation result by the electromotive force estimation method. The vertical axis of the graph of each of the results E1 to E25 represents the potential. The unit of potential is microvolt. The horizontal axis of the graph of each of the results E1 to E25 represents time. The unit of time is millisecond.

In FIGS. 10 to 34, each of time series D201, D203, D205, D207, D209, D211, D213, D215, D217, D219, D221, D223, D225, D227, D229, D231, D233, D235, D237, D239, D241, D243, D245, D247, and D249 is a measured electrocardiographic potential time series.

In FIGS. 10 to 34, each of time series D202, D204, D206, D208, D210, D212, D214, D216, D218, D220, D222, D224, D226, D228, D230, D232, D234, D236, D238, D240, D242, D244, D246, D248, and D250 is an electromotive force time series of the estimation result by the electromotive force estimation method.

The time series D202, D204, D206, D208, D210, D212, D214, D216, D218, D220, D222, D224, D226, D228, D230, D232, D234, D236, D238, D240, D242, D244, D246, D248, and D250 all have features similar to a feature medically recognized as a feature of the electromotive force time series of a person whose myocardium movement is normal. The results E1 to E25 in FIGS. 10 to 34 indicate that the electromotive force estimation method of the embodiment is appropriate as a method of estimating the electromotive force time series of a person whose myocardium movement is normal with high accuracy.

FIG. 35 is a thirty-third explanatory diagram in which the measured electrocardiographic potential time series is used for illustrating the certainty of the estimation result by the electromotive force estimation method of the embodiment. FIG. 35 illustrates a time series D301 and a time series D302. The time series D301 is a measured electrocardiographic potential time series. The time series D302 is an electromotive force time series estimated by the electromotive force estimation method based on the time series D301.

The time series D301 indicates that the premature contraction occurs in the myocardium movement in the period from time T5 to time T6. The fact that the premature contraction occurs in the myocardium movement means that the myocardium movement is abnormal. The time series D301 indicates that the myocardium movement is a sinus rhythm in the other periods. The fact that the myocardium movement is a sinus rhythm means that the myocardium movement is normal.

The time series D302 indicates that a medically known feature appears in the waveform of the electromotive force at the time of premature contraction. In the time series D302, the waveform at the time of premature contraction is a waveform in a period from time T5 to time T6. The feature of the waveform of the electromotive force at the time of premature contraction specifically includes a feature that the length of the plateau phase (that is, the refractory period) is shorter than that in a normal case, and a feature that the start of the repolarization phase (that is, the repolarization period) is earlier than that in a normal case.

As described above, FIG. 35 indicates that the electromotive force estimation method of the embodiment is appropriate as a method of estimating, with high accuracy, the electromotive force time series of a person with the premature contraction.

FIG. 36 is a thirty-fourth explanatory diagram in which the measured electrocardiographic potential time series is used for illustrating the certainty of the estimation result by the electromotive force estimation method of the embodiment. FIG. 36 illustrates a time series D401 and a time series D402. The time series D401 is a measured electrocardiographic potential time series. The time series D402 is an electromotive force time series estimated by the electromotive force estimation method based on the time series D401.

The time series D401 indicates that the premature contraction occurs in the myocardium movement in a period from time T7 to time T8. The time series D401 indicates that the premature contraction occurs in the myocardium movement in the period from time T9 to time T10.

The time series D402 indicates that the waveform in the period from time T7 to time T8 has a feature that the length of the plateau phase is shorter than that in a normal case, and a feature that the start of the repolarization phase is earlier than that in a normal case. The waveform of the time series D402 also indicates that the waveform in the period from time T7 to time T8 is abnormal associated with the atrioventricular block. The abnormality associated with the atrioventricular block appears in the waveform as a feature of a QRS wave and ST heteromorphy due to ectopic stimulation pathways.

The time series D402 indicates that the waveform in the period from time T9 to time T10 has a feature that the length of the plateau phase is shorter than that in a normal case, and a feature that the start of the repolarization phase is earlier than that in a normal case. The waveform of the time series D402 also indicates that the waveform in the period from time T7 to time T8 is abnormal associated with the atrioventricular block. The abnormality associated with the atrioventricular block appears in the waveform as a feature of a QRS wave and ST heteromorphy due to ectopic stimulation pathways.

As described above, the time series D402 indicates that a medically known feature of the waveform of the electromotive force at the time of the premature contraction associated with the atrioventricular block appears in the waveform in the period from the time T7 to the time T8. Further, the time series D402 indicates that a medically known feature of the waveform of the electromotive force at the time of the premature contraction associated with the atrioventricular block appears in the waveform in the period from the time T9 to the time T10.

As described above, FIG. 36 indicates that the electromotive force estimation method of the embodiment is appropriate as a method of estimating, with high accuracy, the electromotive force time series of a person with the premature contraction.

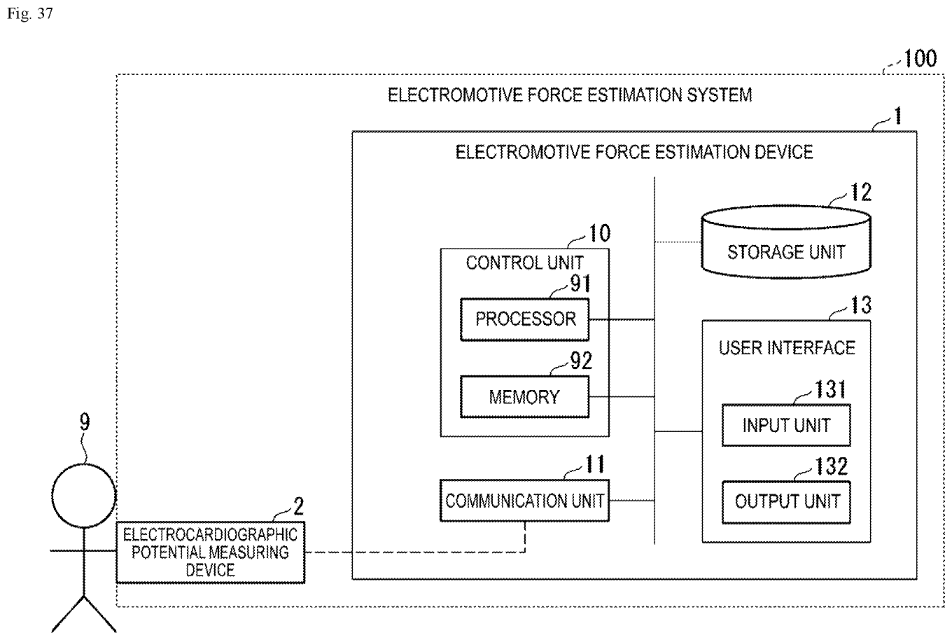

FIG. 37 is a diagram illustrating an example of a configuration of an electromotive force estimation system 100 that executes the electromotive force estimation method of the embodiment. The electromotive force estimation system 100 includes an electromotive force estimation device 1 and an electrocardiographic potential measuring device 2. The electromotive force estimation device 1 acquires the electrocardiographic potential time series measured by the electrocardiographic potential measuring device 2, and estimates the electromotive force time series in the period indicated by the acquired electrocardiographic potential time series based on the acquired electrocardiographic potential time series.

The electrocardiographic potential measuring device 2 measures an electrocardiographic potential to be estimated in the electromotive force time series. The electrocardiographic potential measuring device 2 outputs an electrocardiographic potential time series, which is a time series of the electrocardiographic potential of the measurement result, to the electromotive force estimation device 1. The electrocardiographic potential measuring device 2 is, for example, an electrocardiograph.

The electromotive force estimation device 1 includes a control unit 10 including a processor 91 such as a central processing unit (CPU) connected via a bus and a memory 92, and executes a program. The electromotive force estimation device 1 functions as a device including the control unit 10, a communication unit 11, a storage unit 12, and a user interface 13 by execution of the program.

More specifically, in the electromotive force estimation device 1, the processor 91 reads a program stored in the storage unit 12, and stores the read program in the memory 92. The processor 91 executes the program stored in the memory 92, whereby the electromotive force estimation device 1 functions as a device including the control unit 10, the communication unit 11, the storage unit 12, and the user interface 13.

The control unit 10 controls the operation of each functional unit included in the electromotive force estimation device 1. The control unit 10 controls, for example, the operation of the communication unit 11. The control unit 10 controls, for example, the operation of the communication unit 11 to acquire the electrocardiographic potential time series from the electrocardiographic potential measuring device 2. The control unit 10 records the acquired electrocardiographic potential time series in the storage unit 12.

The control unit 10 estimates the electromotive force time series using, for example, the electromotive force estimation method. More specifically, the control unit 10 acquires the electrocardiographic potential time series input via the communication unit 11 or the user interface 13, and estimates the electromotive force time series in the period indicated by the electrocardiographic potential time series by the electromotive force estimation method based on the acquired electrocardiographic potential time series. The control unit 10 records the acquired electromotive force time series in the storage unit 12.

For example, the control unit 10 controls the operation of the output unit 132 to cause the output unit 132 to output the electromotive force time series of the estimation result.

The communication unit 11 includes a communication interface for connecting the electromotive force estimation device 1 to one or a plurality of external devices including the electrocardiographic potential measuring device 2. One of the external devices is the electrocardiographic potential measuring device 2. The communication unit 11 acquires the electrocardiographic potential time series from the electrocardiographic potential measuring device 2. One of the external devices is, for example, a printer that outputs the electromotive force time series of the estimation result. In such a case, the communication unit 11 transmits the electromotive force time series of the estimation result to the printer that is one of the external devices.

The storage unit 12 includes a storage device such as a magnetic hard disk device or a semiconductor storage device. The storage unit 12 stores various types of information regarding the electromotive force estimation device 1. The storage unit 12 stores, for example, a program for controlling the operation of each functional unit included in the electromotive force estimation device 1 in advance. The storage unit 12 stores, for example, a program for executing the electromotive force estimation method in advance. The storage unit 12 stores, for example, the electrocardiographic potential time series of the estimation result. The storage unit 12 stores, for example, the electromotive force time series of the estimation result.

The user interface 13 includes an input unit 131 that receives an input to the electromotive force estimation device 1 and an output unit 132 that displays various types of information regarding the electromotive force estimation device 1. The user interface 13 is, for example, a touch panel. The input unit 131 receives an input to the electromotive force estimation device 1. The input unit 131 is, for example, an input terminal such as a mouse, a keyboard, or a touch panel. The input unit 131 may be formed as, for example, an interface that connects these input terminals to the electromotive force estimation device 1. The input received by the input unit 131 is, for example, an input instructing start of estimation of the electromotive force. The input received by the input unit 131 may be, for example, an electrocardiographic potential time series.

The output unit 132 is, for example, a display device such as a liquid crystal display or an organic electro luminescence (EL) display. The output unit 132 may be formed as, for example, an interface that connects these display devices to the electromotive force estimation device 1. The output unit 132 may be, for example, a sound output device such as a speaker. The information output by the output unit 132 is, for example, the electromotive force time series of the estimation result. The operation of the output unit 132 is controlled by the control unit 10. The output unit 132 outputs the electromotive force time series of the estimation result according to the instruction of the control unit 10.

The electromotive force estimation method configured as described above classifies pieces of data of the electrocardiographic potential time series into any one of four types of depolarization electrocardiographic potential time series data, refractory electrocardiographic potential time series data, repolarization electrocardiographic potential time series data, and resting electrocardiographic potential time series data for each pulsation.

The electromotive force estimation method estimates a depolarization electromotive force time series based on the depolarization electrocardiographic potential time series obtained as a result of classification. As described above, the depolarization electromotive force time series is data having a depolarized electromotive force estimation value for each time point of the depolarization electrocardiographic potential time series. As described above, the depolarized electromotive force estimation value is a value obtained by adding the sum of past depolarization difference potentials indicated by the depolarization electrocardiographic potential time series to the electrocardiographic potential at the start time point of the depolarization period indicated by the depolarization electrocardiographic potential time series.

The electromotive force estimation method can estimate the depolarization electromotive force time series by calculation with a smaller calculation load than that of an estimation simulation method. The estimation simulation is a method of estimating an electromotive force by solving an inverse problem by simulation using a mathematical model that reproduces electrocardiographic potential time series.

The electromotive force estimation method estimates a refractory electromotive force time series based on the refractory electrocardiographic potential time series obtained as a result of classification. As described above, the refractory electromotive force time series is data having a refractory electromotive force estimation value for each time point of the refractory electrocardiographic potential time series. As described above, the refractory electromotive force estimation value at each time point is a value obtained by subtracting the absolute value of the electrocardiographic potential at the corresponding time point of the refractory electrocardiographic potential time series from the value of the end time point depolarized electromotive force estimation value.

The electromotive force estimation method can estimate the refractory electromotive force time series by calculation with a smaller load than that of the estimation simulation method.

The electromotive force estimation method estimates a repolarization electromotive force time series based on the repolarization electrocardiographic potential time series obtained as a result of classification. As described above, the repolarization electromotive force time series is data having a repolarization electromotive force estimation value for each time point of the repolarization electrocardiographic potential time series. As described above, the repolarization electromotive force estimation value is a value obtained by subtracting the sum of the past repolarization difference potentials indicated by the repolarization electrocardiographic potential time series from the end time point refractory electromotive force estimation value.

The electromotive force estimation method can estimate the repolarization electromotive force time series by calculation with a smaller load than the estimation simulation method.

As described above, in the electromotive force estimation method, the electrocardiographic potential time series is classified into the depolarization period, the refractory period, the repolarization period, and the resting period, and the electromotive force is estimated for each classified data. It is necessary to reproduce the electrocardiographic potential time series in the method of estimating an electromotive force by solving an inverse problem by simulation using a mathematical model that reproduces electrocardiographic potential time series. In the simulation using such a mathematical model, all data of the electrocardiographic potential time series generated in one pulsation is required to estimate the electromotive force at one time point.

On the other hand, in the case of the electromotive force estimation method, not all of the electrocardiographic potential time series are necessarily required to estimate the electromotive force at one time point. For example, the electromotive force in the depolarization period can be estimated only with data of the electromotive force in the depolarization period. As described above, the electromotive force estimation method can estimate the electromotive force time series by calculation with a smaller load than simulation using a mathematical model. The electromotive force estimation method can reduce a calculation load when estimating the electromotive force.

### Modified Example

In the electromotive force estimation method, matching processing may be executed for each unit electromotive force time series indicated by the electromotive force time series. The matching processing is processing of multiplying the electromotive force indicated by the data in the period after the end time point of the repolarization period by a constant so that the average value of the electromotive force indicated by the data in the period after the end time point of the repolarization period substantially matches the average value of the electromotive force indicated by the data before the start time point of the depolarization period.

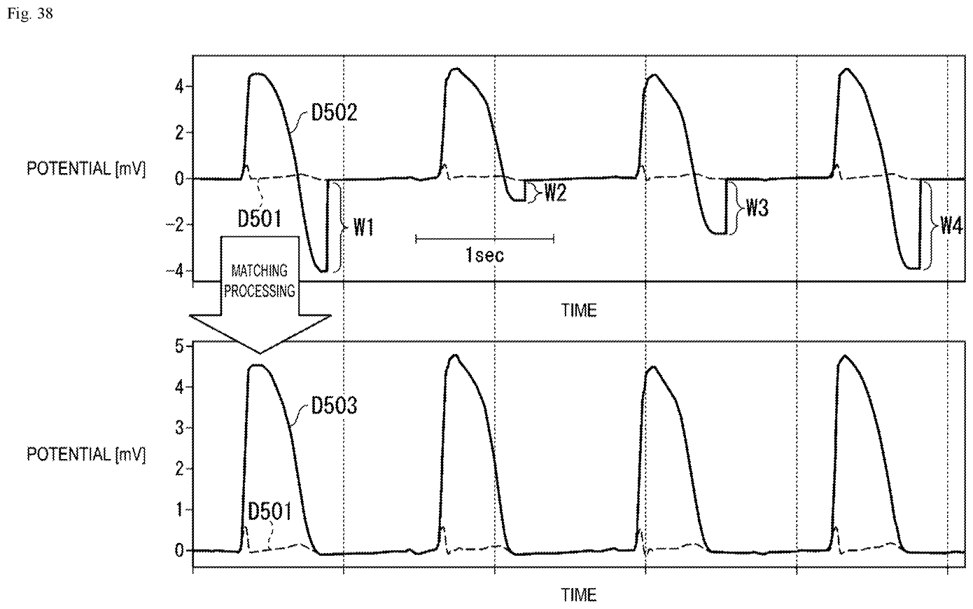

FIG. 38 is an explanatory diagram illustrating an effect of the matching processing in a modified example. FIG. 38 is a diagram illustrating the electromotive force time series in a case where the matching processing is not executed and the electromotive force time series after execution of the matching processing.

FIG. 38 illustrates an electrocardiographic potential time series D501 and an electromotive force time series D502 in the upper graph. The electromotive force time series D502 is an electromotive force time series estimated by the electromotive force estimation method based on the electrocardiographic potential time series D501, and is an electromotive force time series on which matching processing is not executed. The horizontal axis in the upper graph of FIG. 38 represents time. The vertical axis in the upper graph of FIG. 38 represents the potential. The unit of the vertical axis in the upper graph of FIG. 38 is millivolts.

FIG. 38 illustrates an electromotive force time series D503 in the lower graph. The electromotive force time series D503 is an electromotive force time series estimated by the electromotive force estimation method based on the electrocardiographic potential time series D501, and is an electromotive force time series after execution of the matching processing. The horizontal axis in the lower graph of FIG. 38 represents time. The vertical axis in the lower graph of FIG. 38 represents the potential. The unit of the vertical axis in the lower graph of FIG. 38 is millivolts.

The upper graph in FIG. 38 shows the potential difference between the potential of the negative peak of the potential in the electromotive force time series D502 and the potential indicated by the base line. Specifically, a potential difference W1, a potential difference W2, a potential difference W3, and a potential difference W4 in the upper graph of FIG. 38 are potential differences between the potential of the negative peak of the potential in the electromotive force time series D502 and the potential indicated by the base line.

The electromotive force time series D503 illustrated in the lower graph of FIG. 38 indicates that the potential difference between the potential of the negative peak of the potential and the potential indicated by the base line is smaller than that of the electrocardiographic potential time series D502.

As described above, when the matching processing is not performed, the average value of the potentials in the refractory period in the electromotive force time series is not necessarily substantially constant. On the other hand, by executing the matching processing, the electromotive force time series in which the average value of the potentials in the refractory period is substantially constant is acquired.

FIG. 38 also shows that the base line of the electromotive force time series D503 has a higher degree of parallelism with respect to the base line of the electromotive force time series D502 than the base line of the electrocardiographic potential time series D501.

As described above, in the matching processing, the degree of parallelism with respect to the base line of the electrocardiographic potential time series in the electromotive force time series can be made higher than a case where the matching processing is not performed.

As described above, since the average value of the potentials in the refractory period becomes substantially constant by executing the matching processing, the graph of the electromotive force time series becomes a graph that is easy to see for the user. The user can acquire the content indicated by the electromotive force time series with less effort than a case where the matching processing is not performed.

In addition, since the degree of parallelism with respect to the base line of the electrocardiographic potential time series in the electromotive force time series is increased by executing the matching processing, the graph of the electromotive force time series becomes a graph that is easy to see for the user. The user can acquire the content indicated by the electromotive force time series with less effort than a case where the matching processing is not performed.

The processing of acquiring the refractory period electromotive force time series is not limited to the processing described in the present specification. For example, the processing of acquiring the refractory period electromotive force time series may be processing of acquiring a deformed refractory electromotive force estimation value as the electromotive force corresponding to each piece of data of the refractory electrocardiographic potential time series (hereinafter referred to as “refractory electromotive force estimation deformation processing”). For example, the deformed refractory electromotive force estimation value may be a value obtained by adding a value obtained by multiplying the electrocardiographic potential indicated by the refractory electrocardiographic potential time series by a constant to a value of the end time point depolarized electromotive force estimation value. The constant is a value determined according to the height of the repolarization electrocardiographic potential time series. The constant is, for example, 1/100 to 1/200 of the T wave maximum potential. The constant may be adjusted, for example, by the magnitude of the electrocardiographic potential.

The waveform in the refractory period indicated by the electromotive force time series estimated by the electromotive force estimation method including the refractory electromotive force estimation deformation processing is more complicated than the waveform in the refractory period indicated by the electromotive force time series estimated by the electromotive force estimation method of the embodiment. The complicated waveform means that the amount of information is large. The electromotive force time series estimated by the electromotive force estimation method including the refractory electromotive force estimation deformation processing indicates more information regarding the myocardium movement than the electromotive force time series estimated by the electromotive force estimation method of the embodiment.

The method of estimating the refractory electromotive force time series may be a method of estimating the refractory electromotive force time series using a learned model in which the relationship between the refractory electrocardiographic potential time series and the refractory electromotive force time series has been learned in advance by machine learning. In such a case, the explanatory variable of the learned model is the refractory electrocardiographic potential time series, and the dependent variable is the refractory electromotive force time series. The machine learning method may be recursive neural network or deep learning.

The method of estimating the depolarization electromotive force time series is not limited to the method described in the present specification. For example, the method of estimating the depolarization electromotive force time series may be a method of estimating using a bijective mapping indicating a correspondence relationship between the depolarization electromotive force time series and the depolarization electrocardiographic potential time series acquired in advance.

The method of estimating the repolarization period electromotive force time series is not limited to the method described in the present specification. For example, the method of estimating the repolarization period electromotive force time series may be a method of estimating using a bijective mapping indicating a correspondence relationship between the depolarization electromotive force time series and the depolarization electrocardiographic potential time series acquired in advance.

The processing in step S200 is an example of a division step. The processing in step S302 is an example of a state time series acquisition step. The processing in step S303 is an example of a depolarization electromotive force estimation step. The processing in step S304 is an example of a refractory electromotive force estimation step. The processing in step S305 is an example of a repolarization electromotive force estimation step.

Each of the communication unit 11 and the input unit 131 is an example of an acquisition unit. The processing in step S200 is an example of division processing. The processing in step S302 is an example of state time series acquisition processing. The processing in step S303 is an example of depolarization electromotive force estimation processing. The processing in step S304 is an example of refractory electromotive force estimation processing. The processing in step S305 is an example of repolarization electromotive force estimation processing.

The electromotive force estimation device 1 may be implemented using a plurality of information processing devices communicably connected via a network. In this case, the functional units included in the electromotive force estimation device 1 may be distributed and implemented in a plurality of information processing devices.

All or some of the functions of the electromotive force estimation device 1 may be implemented by using hardware such as an application specific integrated circuit (ASIC), a programmable logic device (PLD), a field programmable gate array (FPGA), or physical reserve computing (PRC). The program may be recorded on a computer-readable recording medium. The computer-readable recording medium is, for example, a portable medium such as a flexible disk, a magneto-optical disc, a ROM, or a CD-ROM, or a storage device such as a hard disk incorporated in a computer system. The program may be transmitted via an electrical communication line.

Although the embodiment of the present disclosure has been described in detail with reference to the drawings, a specific configuration is not limited to the embodiment, and a design or the like in a range that does not depart from the gist of the present disclosure is included.

### REFERENCE SIGNS LIST

100 Electromotive force estimation system
1 Electromotive force estimation device
2 Electrocardiographic potential measuring device
10 Control unit
11 Communication unit
12 Storage unit
13 User interface

1. An electromotive force estimation method, comprising:
dividing a time series of an electrocardiographic potential to be estimated in a time series of an electromotive force such that a time series representing an electrocardiographic potential generated by one pulsation of a myocardium is separated from each other;
acquiring, on a unit electrocardiographic potential time series basis, a depolarization electrocardiographic potential time series that is a set of data belonging to a depolarization period, a refractory electrocardiographic potential time series that is a set of data belonging to a refractory period, and a repolarization electrocardiographic potential time series that is a set of data belonging to a repolarization period, by classifying pieces of data of the unit electrocardiographic potential time series into data belonging to the depolarization period, data belonging to the refractory period, data belonging to the repolarization period, and data belonging to a resting period for each unit electrocardiographic potential time series while setting each of the time series divided in the dividing as the unit electrocardiographic potential time series;
estimating a depolarization electromotive force time series indicating an electromotive force at a plurality of time points in the depolarization period based on the depolarization electrocardiographic potential time series;
estimating a refractory electromotive force time series indicating an electromotive force at a plurality of time points of the refractory period based on the refractory electrocardiographic potential time series and an estimation value of the electromotive force at an end time point of the depolarization period; and
estimating a repolarization electromotive force time series indicating an electromotive force at a plurality of time points of the repolarization period based on the repolarization electrocardiographic potential time series and an estimation value of the electromotive force at an end time point of the refractory period.

2. The electromotive force estimation method according to claim 1, wherein when an electromotive force included in the depolarization electromotive force time series is estimated in the estimating of the depolarization electromotive force time series, a value is acquired as the electromotive force, the value being obtained by adding a sum of an absolute value of a difference between an adjacent electrocardiographic potential and another adjacent electrocardiographic potential from a start time point of the depolarization period to a time point reaching the estimated electromotive force to an electrocardiographic potential at the start time point.

3. The electromotive force estimation method according to claim 1, wherein in the estimating of the depolarization electromotive force time series, the depolarization electromotive force time series to be estimated is estimated by using a bijective mapping indicating a correspondence relationship between the depolarization electromotive force time series which has been acquired in advance and the depolarization electrocardiographic potential time series.

4. The electromotive force estimation method according to claim 1, wherein when an electromotive force included in the repolarization electromotive force time series is estimated in the estimating of the repolarization electromotive force time series, a value is acquired as the electromotive force, the value being obtained by subtracting a sum of a plurality of values based on a difference between adjacent electrocardiographic potential and another adjacent electrocardiographic potential from an end time point of the refractory period in the repolarization electrocardiographic potential time series to a time point reaching the estimated electromotive force from an estimation value of the electromotive force at the end time point.

5. The electromotive force estimation method according to claim 1, wherein in the estimating of the repolarization electromotive force time series, the repolarization electromotive force time series to be estimated is estimated by using a bijective mapping indicating a correspondence relationship between the repolarization electromotive force time series which has been acquired in advance and the repolarization electrocardiographic potential time series.

6. The electromotive force estimation method according to claim 1, wherein when an electromotive force included in the refractory electromotive force time series is estimated in the estimating of the refractory electromotive force time series, a value is acquired as the electromotive force, the value being obtained by subtracting a value related to an electrocardiographic potential at a corresponding time point in the refractory period from an estimation value of the electromotive force at an end time point of the depolarization period.

7. The electromotive force estimation method according to claim 1, wherein the electromotive force at the time points estimated in the estimating of the refractory electromotive force time series is a value estimated by using a learned model in which a relationship between the refractory electrocardiographic potential time series and the refractory electromotive force time series has been learned in advance by machine learning.

8. The electromotive force estimation method according to claim 1, wherein when the electromotive force included in the refractory electromotive force time series is estimated in the estimating of the refractory electromotive force time series, a value is acquired as the electromotive force, the value being obtained by adding a value obtained by multiplying an electrocardiographic potential indicated by the refractory electrocardiographic potential time series by a constant to a value of an estimation value of the electromotive force at an end time point of the depolarization period.

9. An electromotive force estimation device, comprising:
an acquisition unit configured to acquire an electrocardiographic potential time series that is a time series of an electrocardiographic potential to be estimated in a time series of an electromotive force; and
a control unit configured to execute:
dividing the electrocardiographic potential time series such that a time series representing electrocardiographic potentials generated by one pulsation of a myocardium are separated from each other;
acquiring, on a unit electrocardiographic potential time series basis, a depolarization electrocardiographic potential time series that is a set of data belonging to a depolarization period, a refractory electrocardiographic potential time series that is a set of data belonging to a refractory period, and a repolarization electrocardiographic potential time series that is a set of data belonging to a repolarization period, by classifying pieces of data of the unit electrocardiographic potential time series into data belonging to the depolarization period, data belonging to the refractory period, data belonging to the repolarization period, and data belonging to a resting period for each unit electrocardiographic potential time series while setting each of the time series divided in the dividing as the unit electrocardiographic potential time series;
estimating a depolarization electromotive force time series indicating an electromotive force at a plurality of time points in the depolarization period based on the depolarization electrocardiographic potential time series;
estimating a refractory electromotive force time series indicating an electromotive force at a plurality of time points of the refractory period based on the refractory electrocardiographic potential time series and an estimation value of the electromotive force at an end time point of the depolarization period; and
estimating a repolarization electromotive force time series indicating an electromotive force at a plurality of time points of the repolarization period based on the repolarization electrocardiographic potential time series and an estimation value of the electromotive force at an end time point of the refractory period.

10. A non-transitory computer-readable medium having computer-executable instructions that, upon execution of the instructions by a processor of a computer, cause the computer to function as the electromotive force estimation device according to claim 9.
